(19) United States
(12) Patent Application Publication
(10) Pub. No.: US 2023/0372252 A1
(43) Pub. Date: Nov. 23, 2023

(54) CONTROLLED RELEASE FILL COMPOSITIONS AND CAPSULES CONTAINING SAME

(71) Applicant: R.P. Scherer Technologies, LLC, Carson City, NV (US)

(72) Inventors: Qi Fang, St. Petersburg, FL (US); Karunakar Sukuru, St. Petersburg, FL (US)

(73) Assignee: R.P. Scherer Technologies, LLC, Carson City, NV (US)

(21) Appl. No.: 18/248,415

(22) PCT Filed: Oct. 14, 2021
(86) PCT No.: PCT/US2021/054991

§ 371 Date: Apr. 10, 2023

Related U.S. Application Data
(60) Provisional application No. 63/092,679, filed on Oct. 16, 2020.

(51) Int. Cl. A61K 9/48; A61K 47/10

(52) U.S. Cl. CPC A61K 9/4833; A61K 47/10

(57) ABSTRACT

A controlled release fill composition for use in soft or hard capsules, soft- or hard-shell capsules encapsulating controlled release fill compositions, a method of producing a softgel capsule with a controlled release fill composition encapsulated in the soft gel capsule shell. The controlled release fill composition includes an active pharmaceutical ingredient; polyethylene oxide having a number average molecule weight of from 0.05 M daltons to 15 M daltons; and at least one of water or a hydrophilic carrier having a number average molecule weight of from 200 daltons to 5000 daltons. Also, in the controlled release fill composition either the polyethylene oxide is present in an amount of at least 21.5 wt. %, based on a total weight of the controlled release fill composition, or the hydrophilic carrier is present in an amount up to 65 wt. %, based on a total weight of the controlled release fill composition.

### BACKGROUND OF THE INVENTION

### Field of the Invention

The present disclosure relates to controlled release fill compositions for encapsulation in capsules, which fill compositions incorporate different types and amounts of controlled release materials (e.g., polyethylene oxide) to control or modify drug release rates. The present disclosure also relates to capsules containing the controlled release fill compositions, and methods for making the capsules and controlled release fill compositions.

### Description of the Related Technology

Capsules are well-known dosage forms that normally include a shell filled with a fill composition containing one or more active pharmaceutical ingredients or other excipients. Soft gelatin capsules (softgel capsules) have been used in the pharmaceutical industry as an important medical dosage form for a long time. A softgel capsule may refer to a solid capsule/shell surrounding a liquid or semi solid inner fill composition having an active ingredient incorporated into the fill composition.

Compared to other medical dosage forms softgel capsules provide advantages including easy swallowing; taste/odor masking; enabling a variety of routes of administration; the convenience of a unit dose; tamper-resistance; a wide variety of colors, shapes, and sizes; the ability to accommodate a wide variety of active ingredients; use for immediate or delayed drug delivery; and a potentially positive influence on the bioavailability of active ingredients incorporated therein.

In some instances, controlled release softgel capsules are needed to deliver drug substances over a prolonged period (typically 8 to 24 hours). Current controlled release softgel products utilize waxy matrix formulations. The fill materials must be kept at high temperatures during capsule encapsulation to maintain the viscosity low enough to facilitate encapsulation. High temperatures may affect thermally sensitive drug substances and the hot fills can adversely affect the gelatin shell potentially influencing one or both of capsule sealing and shape, particularly when the encapsulation temperature exceeds 35-40 ° C.

Polyethylene oxide (PEO) resins have been used in pharmaceutical product development for modified release and abuse deterrent compositions in place of waxy matrix formulations. The use of polyethylene oxide resins can avoid the need for the high temperatures required for material preparation and encapsulation of the waxy matrix materials, since the PEO resins can be encapsulated at lower temperatures of about 20-35° C.

For example, U.S. Pat. No. 9,861,629 discloses an abuse deterrent controlled release oral dosage form and method for producing the same, some of which employed PEO resins. However, attempts at controlling the release rates using PEO led to compositions that increased the difficulty of processing steps needed to make the capsules of this patent and required the inclusion of flowability enhancers, such as glyceryl monolinoleate.

U.S. Pat. No. 8,101,630 also discloses an abuse deterrent dosage form that provides extended release of a pharmaceutical. The dosage form of this disclosure includes PEO resins for increasing the viscosity of the solution in a situation where the dosage form is tampered with by crushing or dissolving it. The dosage form of this disclosure also requires the inclusion of magnesium stearate to facilitate processing.

For many active pharmaceutical ingredients, controlled release of the drug from the capsule fill composition is desirable. Existing controlled release fill compositions for softgel capsules are associated with processing challenges which are sometimes addressed by the incorporation of excipients that facilitate processing. Such excipients may be undesirable and may occupy volume within the fill composition that could otherwise be occupied by a greater dose of an active ingredient. Alternatively, such excipients could be eliminated altogether, thereby enabling production of a smaller capsule per unit dose that is easier to swallow.

Thus, a controlled release capsule fill composition that can be readily encapsulated with minimal use of excipients that facilitate processing is sought.

### SUMMARY OF THE INVENTION

In a first embodiment, the disclosure relates to a controlled release capsule fill composition including:
(i) an active pharmaceutical ingredient;
(ii) polyethylene oxide having a number average molecule weight of from 0.05 M daltons to 15 M daltons; and
(iii) at least one of water or a hydrophilic carrier having a number average molecule weight of from 200 daltons to 5000 daltons,
   wherein either:
(I) the polyethylene oxide is present in an amount of at least 21.5 wt. % of the controlled release fill composition, based on a total weight of the controlled release fill composition; or
(II) the water and/or the hydrophilic carrier is present in an amount of up to 65 wt. % of the controlled release fill composition, based on a total weight of the controlled release fill composition.

The active pharmaceutical ingredient may comprise from about 1 wt. % to about 60 wt. % of the controlled release capsule fill composition, based on a total weight of the controlled release fill composition.

In the controlled release capsule fill composition of each of the foregoing embodiments, the polyethylene oxide may comprise from 10 wt. % to 65 wt. % of the controlled release fill composition, based on a total weight of the controlled release fill composition.

In the controlled release capsule fill composition of each of the foregoing embodiments, the water and/or the hydrophilic carrier may comprise from about 30 wt. % to about 70 wt. % of the controlled release fill composition, based on a total weight of the controlled release fill composition.

In the controlled release capsule fill composition of each of the foregoing embodiments, the number average molecule weight of the polyethylene oxide is from 900,000 to 7,000,000 Daltons.

In the controlled release capsule fill composition of each of the foregoing embodiments, the hydrophilic carrier may comprise from 40-60 wt. % of the controlled release fill composition, based on a total weight of the controlled release fill composition.

In the controlled release capsule fill composition of each of the foregoing embodiments, the hydrophilic carrier may be selected from the group consisting of polyethylene glycol, polypropylene glycol, and other hydrophilic solvents.

In the controlled release capsule fill composition of each of the foregoing embodiments, the polyethylene oxide may comprise from 25-40 wt. % of the fill composition, based on a total weight of the fill composition.

In an embodiment, the disclosure relates to a controlled release capsule fill composition including:
(i) an active pharmaceutical ingredient that is not susceptible to abuse;
(ii) polyethylene oxide; and
(iii) at least one of water or a hydrophilic carrier.

In an embodiment, the disclosure relates to a controlled release capsule fill composition including:
(i) an active pharmaceutical ingredient;
(ii) polyethylene oxide; and
(iii) at least one of water or a hydrophilic carrier, wherein the weight to weight ratio of (ii) to (iii) ranges from about 10:1 up to 1:3.

In another embodiment, the present invention encompasses a capsule including:
(a) a softgel capsule shell or a hard capsule shell; and
(b) the controlled release fill composition of any of the foregoing embodiments encapsulated in the softgel capsule shell or hard capsule shell.

In an embodiment, the present invention encompasses a capsule including:
(a) a softgel gelatin capsule shell; and
(b) the controlled release fill composition of any of the foregoing embodiments encapsulated in the softgel gelatin capsule shell.

In an embodiment, the present invention encompasses an annealed capsule including:
(a) a softgel capsule shell or a hard capsule shell; and
(b) the controlled release fill composition of any of the foregoing embodiments encapsulated in the softgel gelatin capsule shell.

In the capsule of the foregoing embodiment, less than 80% of the active pharmaceutical ingredient may be released after 0.5 hours in a fiberoptic dissolution test using USP Apparatus II at a paddle speed of 100 rpm at 37° C. in 500 ml of 0.1 N HCl or water.

In a further embodiment, the disclosure relates to a method for producing a capsule. The method includes the steps of (a) mixing a liquid fill composition including:
(i) an active pharmaceutical ingredient;
(ii) polyethylene oxide having a number average molecule weight from about 0.05 M daltons to about 15 M daltons;
(iii) optionally one or more additional release rate controlling polymers, and
(iv) at least one of water or a hydrophilic carrier having a number average molecule weight from 200 daltons to 5000 daltons, wherein either:
(I) the polyethylene oxide is present in an amount of at least 21.5 wt. %, based on a total weight of the fill composition; or
(II) The hydrophilic carrier is present in an amount up to 65 wt. %, based on a total weight of the fill composition.
(b) encapsulating the mixed liquid fill composition in a capsule shell to produce the capsule; and
(c) heating the capsule (which may have been dried after encapsulation in certain embodiments) to a temperature of from about 40° C. to about 80° C. for a period from about 10 minutes to about 180 minutes to form a solid or semi-solid fill composition inside said capsule.

In a further embodiment, the disclosure relates to a method for producing a capsule. The method includes the steps of (a) mixing a liquid fill composition including:
(i) an active pharmaceutical ingredient that is not susceptible to abuse;
(ii) polyethylene oxide;
(iii) optionally one or more additional release rate controlling polymers, and
(iv) at least one of water or a hydrophilic carrier;
(b) encapsulating the mixed liquid fill composition in a capsule shell to produce the capsule; and
(c) heating the capsule (which may have been dried after encapsulation in certain embodiments) to a temperature of from about 40° C. to about 80° C. for a period from about 10 minutes to about 180 minutes to form a solid or semi-solid fill composition inside said capsule.

In yet a further embodiment, the disclosure relates to a method for producing a capsule. The method includes the steps of (a) mixing a liquid fill composition including:
(i) an active pharmaceutical ingredient;
(ii) polyethylene oxide;
(iii) optionally one or more additional release rate controlling polymers, and
(iv) at least one of water or a hydrophilic carrier, wherein the weight to weight ratio of (ii) to (iv) ranges from about 10:1 up to 1:3;
(b) encapsulating the mixed liquid fill composition in a capsule shell to produce the capsule; and
(c) heating the capsule (which may have been dried after encapsulation in certain embodiments) to a temperature of from about 40° C. to about 80° C. for a period from about 10 minutes to about 180 minutes to form a solid or semi-solid fill composition inside said capsule.

In yet a further embodiment, the disclosure relates to a method for producing a softgel gelatin capsule. The method includes the steps of (a) mixing a liquid fill composition including:
(i) an active pharmaceutical ingredient;
(ii) polyethylene oxide;
(iii) optionally one or more additional release rate controlling polymers, and
(iv) at least one of water or a hydrophilic carrier,
(b) encapsulating the mixed liquid fill composition in a gelatin capsule shell to produce the softgel gelatin capsule; and
(c) heating the softgel gelatin capsule (which may have been dried after encapsulation in certain embodiments) to a temperature of from about 40° C. to about 80° C. for a period from about 10 minutes to about 180 minutes to form a solid or semi-solid fill composition inside said softgel gelatin capsule.

In the forgoing embodiment of the method, the active pharmaceutical ingredient may be included in an amount of from about 1 wt. % to about 60 wt. % of the controlled release fill composition, based on a total weight of the controlled release fill composition.

In each of the forgoing embodiments of the method, the polyethylene oxide may be included in the controlled release fill composition in an amount from 10 wt. % to 65 wt. % of the controlled release fill composition, based on a total weight of the controlled release fill composition.

In each of the foregoing methods, the fill composition may further comprise the one or more release rate controlling polymers.

In each of the forgoing embodiments of the method, the water and/or hydrophilic carrier may be included in the controlled release fill composition in an amount from about 30 wt. % to about 70 wt. %, or an amount from about 40 wt. % to about 60 wt. % of the controlled release fill composition, based on a total weight of the controlled release fill composition.

In each of the forgoing embodiments of the method, the polyethylene oxide may be included in the controlled release fill composition in an amount from about 25 wt. % to about 40 wt. % of the fill composition, based on a total weight of the fill composition.

In each of the forgoing embodiments, the active pharmaceutical ingredient may be an active pharmaceutical ingredient that is classified in one of the Biopharmaceutics Classification System Classes I, II, III and IV.

In another embodiment, the disclosure relates a softgel capsule or hard capsule made by any of the foregoing methods. In this embodiment of the softgel capsule or hard capsule, less than 80% of the active pharmaceutical ingredient may be released after 0.5 hours in a fiberoptic dissolution test using USP Apparatus II using a paddle speed of 100 rpm at 37° C. in 500 ml of 0.1 N HCl or water.

### BRIEF DESCRIPTION OF THE DRAWINGS

FIG. 1 is a flow diagram showing the steps of a method for manufacturing a capsule according to the disclosure.

FIG. 2 depicts the dissolution profiles of capsules, according to embodiments, obtained in a fiberoptic dissolution test using USP Apparatus II using a paddle speed of 100 RPM at 37° C. in 500 ml of water run at 100 RPM.

FIG. 3 depicts the dissolution profiles of capsules, according to embodiments, obtained in a fiberoptic dissolution test using USP Apparatus II using a paddle speed of 50 RPM at 37° C. in 500 ml of water.

FIGS. 4A-4D and 5-6 show the residual plots for time 90% (hours) for the statistical analysis of dissolution data of Examples 1-6.

FIG. 4A is a normal probability plot for time to release 90% (hours).

FIG. 4B is a versus fits plot for time to release 90% (hours).

FIG. 4C is a histogram for time to release 90% (hours).

FIG. 4D is a versus order plot for time to release 90% (hours).

FIG. 5 is an interaction plot for time to release 90% (hours).

FIG. 6 is a main effects plot for time to release 90% (hours).

FIG. 7 depicts the dissolution profiles for capsules, according to embodiments, filled with formulations 13-15 obtained in a fiberoptic dissolution test using USP Apparatus II using a paddle speed of 100 RPM at 37 ° C. in 500 ml of water.

FIG. 8 depicts the dissolution profiles for capsules, according to embodiments, obtained in a fiberoptic dissolution test using USP Apparatus II using a paddle speed of 100 RPM at 37° C. in 500 ml of water.

FIG. 9 depicts the dissolution profiles for capsules, according to embodiments, obtained in a fiberoptic dissolution test using USP Apparatus II using a paddle speed of 50 RPM at 37° C. in 500 ml of water.

FIG. 10 is a DSC curve of heat flow vs temperature for a capsule fill composition containing polyethylene oxide having a number average molecular weight of 900,000 Da.

FIG. 11 is a DSC curve of heat flow vs temperature for a capsule fill composition containing the MC18-30 fill mix.

FIG. 12 is a DSC curve of heat flow vs temperature for a capsule fill composition containing polyethylene oxide having a number average molecular weight of 5,000,000 Da.

FIG. 13 is a DSC curve of heat flow vs temperature for a capsule fill composition containing the MC18-31 fill mix.

FIG. 14 is a DSC curve of heat flow vs temperature for a capsule fill composition containing polyethylene oxide having a number average molecular weight of 7,000,000 Da.

FIG. 15 is a DSC curve of heat flow vs temperature for a capsule fill composition containing the MC18-32 fill mix.

### DETAILED DESCRIPTION OF THE PREFERRED EMBODIMENT(S)

For illustrative purposes, the principles of the present invention are described by referencing various exemplary embodiments. Although certain embodiments of the invention are specifically described herein, one of ordinary skill in the art will readily recognize that the same principles are equally applicable to, and can be employed in, other systems and methods. Before explaining the disclosed embodiments of the present invention in detail, it is to be understood that the invention is not limited in its application to the details of any embodiment shown. Additionally, the terminology used herein is for the purpose of description and not for limitation. Furthermore, although certain methods are described with reference to steps that are presented herein in a certain order, in many instances, these steps can be performed in any order as may be appreciated by one skilled in the art; the novel method is therefore not limited to the particular arrangement of steps disclosed herein.

It must be noted that as used herein and in the appended claims, the singular forms “a”, “an”, and “the” include plural references unless the context clearly dictates otherwise. Furthermore, the terms “a” (or “an”), “one or more”, and “at least one” can be used interchangeably herein. The terms “comprising”, “including”, “having” and “constructed from” can also be used interchangeably.

Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. As used herein, “about” refers to any values that are within a variation of ±10%, such that “about 10” would include from 9 to 11.

It is to be understood that each component, compound, substituent or parameter disclosed herein is to be interpreted as being disclosed for use alone or in combination with one or more of each and every other component, compound, substituent or parameter disclosed herein.

It is also to be understood that each amount/value or range of amounts/values for each component, compound, substituent or parameter disclosed herein is to be interpreted as also being disclosed in combination with each amount/value or range of amounts/values disclosed for any other component(s), compounds(s), substituent(s) or parameter(s) disclosed herein and that any combination of amounts/values or ranges of amounts/values for two or more component(s), compounds(s), substituent(s) or parameters disclosed herein are thus also disclosed in combination with each other for the purposes of this description.

It is further understood that each lower limit of each range disclosed herein is to be interpreted as disclosed in combination with each upper limit of each range disclosed herein for the same component, compounds, substituent or parameter. Thus, a disclosure of two ranges is to be interpreted as a disclosure of four ranges derived by combining each lower limit of each range with each upper limit of each range. A disclosure of three ranges is to be interpreted as a disclosure of nine ranges derived by combining each lower limit of each range with each upper limit of each range, etc. Furthermore, specific amounts/values of a component, compound, substituent or parameter disclosed in the description or an example is to be interpreted as a disclosure of either a lower or an upper limit of a range and thus can be combined with any other lower or upper limit of a range or specific amount/value for the same component, compound, substituent or parameter disclosed elsewhere in the application to form a range for that component, compound, substituent or parameter.

All references to “molecular weight” herein refer to number average molecular weights unless otherwise specified.

The term “ambient temperature” as used herein refers to a temperature of about 20-35° C.

The present fill composition and method are designed for use in both hard capsules and softgel capsules to provide controlled release of the active pharmaceutical ingredient and provision of abuse resistance. The present fill compositions are liquids at the time of encapsulation in the capsule to make it easier to handle the fill compositions in the capsule filling process. Suitable liquids include solutions, suspensions and dispersions of the ingredients in water and/or a hydrophilic carrier.

The term “softgel capsule” refers to gelatin-containing soft capsules, as well as other types of soft capsules that do not contain gelatin. Similar testing can be used for capsules that do not contain gelatin in order to determine the manufacturing parameters necessary for a particular capsule formulation. “Soft capsule,” “softgel capsule,” and “soft elastic capsule” as used throughout the description refers to capsules that contain gelatin, or other polymer(s) in combination with an explicit plasticizer such as glycerin, PEG 400, or an intrinsic plasticizer such as water.

The term “shell composition” may be used interchangeably with the terms “film composition,” “shell,” and “film” throughout the description. These terms refer to the outer portion of the capsule which encapsulates a fill material.

The term “fill material” may be used interchangeably with the terms “fill composition,” and “fill” throughout the description. These terms refer to the inner portion of the capsule that is encapsulated by the shell composition.

The term, “controlled release” refers to “modified release”, “delayed release” and “extended release” and indicates that the release of the active pharmaceutical ingredient from the fill composition or capsule is controlled to delay, modify or extend the release of the active pharmaceutical ingredient from the fill composition or capsule. In one embodiment, “controlled release” refers to a drug release rate from the controlled release fill composition or the capsule such that less than 80% of the API is released after 0.5 hours in a fiberoptic dissolution test using USP Apparatus II using a paddle speed of 100 RPM at 37° C. in 500 ml of biological, artificial, or simulated gastric fluid, such as 0.1 N HCl and/or biological, artificial, or simulated intestinal fluid, such as pH 6.8 phosphate buffer and/or water. In one embodiment, “controlled release,” refers to an active agent that is released gradually over a period of time, e.g., from about 2 hours to about 24 hours, to provide, for example, a once daily or twice daily dosage form. Controlled release can be important for potent, low dose drugs or for drugs that function better when administered in a controlled manner over time rather than by intermittent dosing.

In one aspect, the present invention relates to controlled release fill compositions suitable for use in capsules (e.g., softgel capsules), which fill compositions contain polyethylene oxide (PEO) resins. Polyethylene oxide polymer is used in the fill compositions to form solid or semi-solid matrices for controlling the release of the active pharmaceutical ingredient (API) from capsules containing such fill compositions. Water and/or hydrophilic carriers may also be included in these fill compositions. By manipulation of the number average molecular weight and/or the concentration of PEO in the fill compositions, the release rate of the API(s) in the fill composition can be controlled.

The process used to produce the capsules (e.g., softgel capsules) may also impact the API release profile. Controlled release materials that are solid or semi-solid at ambient temperature require heating for ease of processing. However, gelatin-based shell materials are sensitive to heat. Therefore, including controlled release materials that require heating for ease of processing are undesirable for use with gelatin-based shell materials. Instead, the present invention, in certain embodiments, fills such gelatin-based capsules with liquid fill compositions at ambient temperatures of about 20-35° C., and subsequently heats the filled capsule to solidify the liquid fill composition to form a solid or semi-solid (and form a polymer matrix), without harming the integrity of the heat sensitive gelatin-based shell materials.

In one embodiment, a fill composition is provided as a mixture containing an API, a hydrophilic carrier and/or water, and a PEO polymer. This mixture may then be encapsulated in a capsule (e.g., softgel capsule) at ambient temperatures of about 20-35° C., which provides the flexibility of using a larger variety of capsule shell materials.

The fill composition may optionally include other components, such as high molecular weight polyethylene oxides and cellulose derivatives. These optional components can be included for a variety of reasons one of which may be to alter the API release profile of the fill composition. The fill composition may also include other additional ingredients, including one or more additional APIs, release rate controlling polymers, inactive ingredients (e.g., pharmaceutically acceptable excipients), or other components of fill compositions for capsules (e.g., softgel capsules) that are known in the art. In certain embodiments, the fill composition may be free or substantially free of flowability enhancing materials such as glyceryl monolinoleate, glyceryl monocaprylate, glyceryl monocaprylcaprate, glyceryl monolinoleate, oleic acid, processability facilitating materials such as magnesium stearate, and the like. Materials that facilitate flowability or processability of the fill composition are merely optional in the instant disclosure because the fill composition is a liquid during processing and, if desired, may solidify into a solid or a semi-solid, after it is already encapsulated within the shell of the capsule.

As used herein, “free or substantially free” of a component, refers to a composition that comprises less than about 1 wt. %, less than about 0.5 wt. %, less than about 0.25 wt. %, less than about 0.1 wt. %, less than about 0.05 wt. %, less than about 0.01 wt. %, or 0 wt. % of said component.

The API may be a pharmaceutical component for therapeutic use. The API can be a single ingredient or a mixture of one or more active pharmaceutical ingredients, as is known in the art, including but limited to any drug, therapeutically acceptable drug salt, drug derivative, drug analog, drug homologue, or polymorph. Preferably, the API is classified in one of the Biopharmaceutics Classification System Classes I, II, III, or IV. The API may encompass APIs that are susceptible to abuse and APIs that are not susceptible to abuse. In one embodiment, the API in the fill composition is susceptible to abuse. In one embodiment, the API in the fill composition is not susceptible to abuse.

Any pharmaceutically active ingredient may be used for purposes of the present disclosure, including both those that are water-soluble and those that are poorly soluble in water. Suitable pharmaceutically active ingredients include, without limitation, analgesics and anti-inflammatory agents (e.g., ibuprofen, naproxen sodium, aspirin), antacids, anthelmintic, anti-arrhythmic agents, anti-bacterial agents, anti-coagulants, anti-depressants, anti-diabetics, anti-diarrheal, anti-epileptics, anti-fungal agents, anti-gout agents, anti-hypertensive agents, anti-malarial, anti-migraine agents, anti-muscarinic agents, anti-neoplastic agents and immunosuppressants, anti-protozoal agents, anti-rheumatics, anti-thyroid agents, anti-histamines (e.g., diphenhydramine), antivirals, anxiolytics, sedatives, hypnotics and neuroleptics, beta-blockers, cardiac inotropic agents, corticosteroids, cough suppressants, cytotoxics, decongestants, diuretics, enzymes, anti-parkinsonian agents, gastro-intestinal agents, histamine receptor antagonists, lipid regulating agents, local anesthetics, neuromuscular agents, nitrates and anti-anginal agents, nutritional agents, opioid analgesics, anticonvulsant agents (e.g., valproic acid), oral vaccines, proteins, peptides and recombinant drugs, sex hormones and contraceptives, spermicides, stimulants, and combinations thereof.

In some embodiments, the active pharmaceutical ingredient may be selected, without limitations, from the group consisting of dabigatran, dronedarone, ticagrelor, iloperidone, ivacaftor, midostaurine, asimadoline, beclomethasone, apremilast, sapacitabine, linsitinib, abiraterone, vitamin D analogs (e.g., calcifediol, calcitriol, paricalcitol, doxercalciferol), COX-2 inhibitors (e.g., celecoxib, valdecoxib, rofecoxib), tacrolimus, testosterone, lubiprostone, pharmaceutically acceptable salts thereof, and combinations thereof.

In one embodiment of the present invention, the active pharmaceutical ingredient is a pain medication such as ibuprofen or an opioid. The term “opioid” refers to a psychoactive compound that works by binding to opioid receptors. Opioids are commonly used in the medical field for their analgesic effects. Opioids are believed to be APIs susceptible to abuse. Examples of opioids include codeine, tramadol, anileridine, prodine, pethidine, hydrocodone, morphine, oxycodone, methadone, diamorphine, hydromorphone, oxymorphone, 7-hydroxymitragynine, buprenorphine, fentanyl, sufentanil, levorphanol, meperidine, tilidine, dihydrocodeine, dihydromorphine, and pharmaceutically acceptable salts thereof.

Examples of the active pharmaceutical ingredient may include N-{1-[2-(4-ethyl-5-oxo-2-tetrazolin-1-yl)ethyl1-4-methoxymethyl-4-piperidyl}propionanilide; alfentanil; 5,5-diallylbarbituric acid; allobarbital; allylprodine; alphaprodine; 8-chloro-1-methyl-6-phenyl-4H-[1,2,4]triazolo[4,3-a][1,4]-benzodiazepine; alprazolam; 2-diethylaminopropiophenone; amfepramone, (±)-αmethylphenethylamine; amphetamine; 2-(α-methylphenethylamino)-2-phenylacetonitrile; amphetaminil; 5-ethyl-5-isopentylbarbituric acid; amobarbital; anileridine; apocodeine; 5,5-diethylbarbituric acid; barbital; benzylmorphine; bezitramide; 7-bromo-5-(2-pyridyl)-1H-1,4-benzodiazepine-2(3H)-one; bromazepam; 2-bromo-4-(2-chlorophenyl)-9-methyl-1-6H-thieno[3,2-f][1,2,4]triazolo[4,3-a][1,4]diazepine; brotizolam, 17-cyclopropylmethyl-4,5a-epoxy-7a[(S)-1-hydroxy-1,2,2-trimethyl-propyl]-6-methoxy-6,14-endo-ethanomorphinan-3-ol; buprenorphine; 5-butyl-5-ethylbarbituric acid; butobarbital; butorphanol; (7-chloro-1,3-dihydro-1-methyl-2-oxo-5-phenyl-2H-1,4-benzodiazepin-3-yl)dimethylcarbamate; camazepam; (1S,2S)-2-amino-1-phenyl-1-propanol; cathine; d-norpseudoephedrine; 7-chloro-N-methyl-5-phenyl-3H-1,4-benzodiazepin-2-yl-amine 4-oxide; chlordiazepoxide, 7-chloro-1-methyl-5-phenyl-1H-1,5-benzodi-azepine-2,4(3H,5H)-dione; clobazam, 5-(2-chlorophenyl)-7-nitro-1H-1,4-benz-odiazepin-2(3H)-one; clonazepam; clonitazene; 7-chloro-2,3-dihydro-2-oxo-5-phenyl-1H-1,4-benzodiazepine-3-carboxylic acid; clorazepate; 5-(2-chlorophenyl)-7-ethyl-1-methyl-1H-thieno[2,3-e][1,4]diazepin-2(3H)-one; clotiazepam; 10-chloro-11b-(2-chlorophenyl)-2,3,7,11b-tetrahydrooxazol-o [3,2-d][1,4]benzodiazepin-6(5H)-one; cloxazolam; (−)-methyl-[3β-benzoyloxy-2β(1αH,5αH)-tropane carboxylate]; cocaine; (5α,6α)-7,8-didehydro-4,5-epoxy-3-methoxy-17-methylmorphinan-6-ol; 4,5α-epoxy-3-methoxy-17-methyl-7-morphinen-6α-ol; codeine; 5-(1-cyclohexenyl)-5-ethyl barbituric acid; cyclobarbital; cyclorphan; cyprenorphine; 7-chloro-5-(2-chloropheny-1)-1H-1,4-benzodiazepin-2(3H)-one; delorazepam; desomorphine; dextromoramide; (+)-(1-benzyl-3-dimethylamino-2-methyl-1-phenylpropyl)propionate; dextropropoxyphene; dezocine; diampromide; diamorphone; 7-chloro-1-methyl-5-phenyl-1H-1,4-benzodiazepin-2(3H)-on; diazepam; 4,5α-epoxy-3-methoxy-17-methyl-6α-morphinanol; dihydrocodeine; 4,5α-epoxy-17-methyl-3,6a-morphinandiol; dihydromorphine; dimenoxadol; dimephetamol; dimethylthiambutene; dioxaphetyl butyrate; dipipanone; (6aR,10aR)-6,6,9-trimethyl-3-pentyl-6a,7,8,10a-tetrahydro-6H-benzo[c]chromen-1-ol; dronabinol; eptazocine; 8-chloro-6-phenyl-4H-[1,2,4]-triazolo[4,3-(a)][1,4]benzodiazepine; estazolam; ethoheptazine; ethylmethylthiambutene; ethyl[7-chloro-5-(2-fluorophenyl)-2,3 -dihydro-2-oxo-1H-1,4-benzodiazepine-3 -c arboxylate] ; ethyl loflazepate; 4,5α-epoxy-3-ethoxy-17-methyl-7-morphinen-6α-ol; ethylmorphine; etonitazene; 4,5 α-epoxy-7α-(1-hydroxy-1-methylbutyl)-6-methoxy-17-methyl-6,14-endo-etheno-morphinan-3 -ol; etorphine; N-ethyl-3 -phenyl- 8,9,10-trinorbornan-2-ylamine; fencamfamine; 7-[2-(α-methylphenethylamino)ethyl]-theophylline; fenethylline; 3-(α-methylphenethylamino)propionitrile; fenproporex; N-(1-phenethyl-4-piperidyl)propionanilide; fentanyl; 7-chloro-5-(2-fluorophenyl)-1-methyl-1H-1,4-benzodiazepin-2(3H)-one; fludiazepam; 5-(2-fluorophenyl)-1-methyl-7-nitro-1H-1,4-benzodiazepin-2(3H)-one; flunitrazepam; 7-chloro-1-(2-diethylaminoethyl)-5-(2-fluorophenyl)-1H-1,4-benzodiazepin-2(3H)-one; flurazepam; 7-chloro-5-phenyl-1-(2,2,2-trifluoroethyl)-1H-1,4-benzodiazepin-2(3H)-one; halazepam; 10-bromo-11b-(2-fluorophenyl)-2,3,7,11b-tetrahydro[1,3]oxazolyl[3,2-d][1,4]benzodiazepin-6(5H)-one; haloxazolam; heroin; 4,5α-epoxy-3-methoxy-17-methyl-6-morphinanone; hydrocodone; 4,5α-epoxy-3-hydroxy-17-methyl-6-morphinanone; hydromorphone; hydroxypethidine; isomethadone; hydroxymethylmorphinan; 11-chloro-8,12b-dihydro-2,8-dimethyl-12b-phenyl-4H-[1,3]oxazino[3,2d][1,4]benzodiazepine-4,7(6H)-dione; ketazolam; 1-[4-(3-hydroxyphenyl)-1-methyl-4-piperidyl]-1-propanone; ketobemidone; (3S,6S)-6-dimethylamino-4,4-diphenylheptan-3 -yl acetate; levacetylmethadol; LAAM; (−)-6-dimethylamino-4,4-diphenol-3-heptanone; levomethadone; (−)-17-methyl-3-morphinanol; levorphanol; levophenacylmorphane; lofentanil; 6-(2-chlorophenyl)-2-(4-methyl-1-piperazinylmethylene)-8-nitro-2H-imidazo [1,2-a][1,4]-benzodiazepin-1(4H)- one; loprazolam; 7-chloro-5-(2-chlorophenyl)-3-hydroxy-1H-1,4-benzodiazepin-2(3H)-one; lorazepam; 7-chloro-5 -(2-chlorophenyl)-3-hydroxy-1-methyl-1H-1,4-benzodiazepin-2(3H)-one; lormetazepam; 5-(4-chlorophenyl)-2,5-dihydro-3H-imidazo[2,1a]isoindol-5-ol; mazindol; 7-chloro-2,3-dihydro-1-methyl-5-phenyl-1H-1,4-benzodiazepine; medazepam; N-(3-chloropropyl)-α-methylphenethylamine; mefenorex; meperidine; 2-methyl-2-propyltrimethylene dicarbamate; meprobamate; meptazinol; metazocine; methylmorphine; N,α-dimethylphenethylamine; metamphetamine; (±)-6-dimethylamino-4,4-diphenol-3-heptanone; methadone; 2-methyl-3-o-tolyl-4(3H)-quinazolinone; methaqualone; methyl 12-phenyl-2-(2-piperidyl)acetatel; methylphenidate; 5-ethyl-1-methyl-5-phenylbarbituric acid; methylphenobarbital; 3,3-diethyl-5-methyl-2,4-piperidinedione; methyprylon; metopon; 8-chloro-6-(2-fluorophenyl)-1-methyl-4H-imidazo[1,5-a][1,4]benzodiazepine; midazolam; 2-(benzhydrylsulfinyl)acetamide; modafinil; (5α,6α)-7,8-didehydro-4,5-epoxy-17-methyl-7-methylmorphinan-3,6-diol; morphine; myrophine; (±)-trans-3-(1,1-dimethylheptyl)-7,8,10,10α-tetrahydro-1-hydroxy-6,6-dimethyl-6H-dibenzo-[b,d]pyran-9 (6 αH)one; nabilone; nalbuphene; nalorphine; narceine; nicomorphine; 1-methyl-7-nitro-5-phenyl-1H-1,4-benzodiazepin-2(3H)-one; nimetazepam; 7-nitro-5-phenyl-1H-1,4-benzodiazepin-2(3H)-one; nitrazepam; 7-chloro-5-phenyl-1H-1,4-benzodiazepin-2(-3H)-one; nordazepam; norlevorphanol; 6-dimethylamino-4,4-diphenyl-3-hexanone; normethadone; normorphine; norpipanone; opium; 7-chloro-3-hydroxy-5-phenyl-1H-1,4-benzodiazepin-2(3H)-one; oxazepam; (cis-/trans-)-10-chloro-2,3,7,11b-tetrahydro-2-methyl-11b-phenyloxazolo[3,2-d][1,4]benzodiazepin-6-(5H)-one; oxazolam; 4,5α-epoxy-14-hydroxy-3-methoxy-17-methyl-6-morphinanone; oxycodone; oxymorphone; papaveretum; 2-imino-5-phenyl-4-oxazolidinone; pemoline; 1,2,3,4,5,6-hexahydro-6,11-dimethyl-3-(3-methyl-2-butenyl)-2,6-methano-3-benzazocin-8-ol; pentazocine; 5-ethyl-5-(1-methylbutyl)-barbituric acid; pentobarbital; ethyl-(1-methyl-4-phenyl-4-piperidinecarboxylate); pethidine; phenadoxone; phenomorphane; phenazocine; phenoperidine; piminodine; pholcodeine; 3-methyl-2-phenylmorpholine; phenmetrazine; 5-ethyl-5-phenylbarbituric acid; phenobarbital; α,α-dimethylphenethylamine; phentermine; (R)-3-[-1-hydroxy-2-(methylamino)ethyl]phenol; phenylephrine, 7-chloro-5-phenyl-1-(2-propynyl)-1H-1,4-benzodiazepin-2(3H)-one; pinazepam; α-(2-piperidyl)benzhydryl alcohol; pipradrol; 1′-(3-cyano-3,3-diphenylpropyl)[1,4′-bipiperidine]-4′-carboxamide; piritramide; 7-chloro-1-(cyclopropylmethyl)-5-phenyl-1H-1,4-benzodiazepin-2(3H)-one; prazepam; profadol; proheptazine; promedol; properidine; propoxyphene; N-(1-methyl-2-piperidinoethyl)-N-(2-pyridyl)propionamide; methyl{3-[4-methoxycarbonyl-4-(N-phenylpropanamido)piperidino]propanoate}; (S,S)-2-methylamino-1-phenylpropan-1-ol; pseudoephedrine, remifentanil; 5-sec-butyl-5-ethylbarbituric acid; secbutabarbital; 5-allyl-5-(1-methylbutyl)-barbituric acid; secobarbital; N-{4-methoxymethyl-1-[2-(2-thienyl)ethyl]-4-piperidyl}propionanilide; sufentanil; 7-chloro-2-hydroxymethyl-5-phenyl-1H-1,4-benzodiazepin-2(3H)-one; temazepam; 7-chloro-5-(1-cyclohexenyl)-1-methyl-1H-1,4-benzodiazepin-2(3H)-one; tetrazepam; ethyl (2-dimethylamino-1-phenyl-3-cyclohexene-1-carboxylate; cis-/trans-tilidine; tramadol; 8-chloro-6-(2-chlorophenyl)-1-methyl-4H-[1,2,4]triazolol4,3-a][1,4]benzodiazepine; triazolam; 5-(1-methylbutyl)-5-vinylbarbituric acid; vinylbital; (1R*,2R*)-3-(3-dimethylamino-1 -ethyl-2-methylpropyl)phenol ; (1R,2R,4S)-2-(dimethylamino)methyl-4-(p-fluorobenzyloxy)-1-(m- methoxyphenyl)cyclohexanol.

In addition to the above compounds, active pharmaceutical ingredients also include a prodrug of any of these compounds. The term “prodrug” means a compound that is a metabolic precursor to the active pharmaceutical ingredient. This precursor is transformed in vivo to provide the active pharmaceutical ingredient which has the desired therapeutic effect.

Active pharmaceutical ingredients also include pharmaceutically acceptable salts of any of the above-mentioned compounds. The phrase “pharmaceutically acceptable salt” of a compound means a salt that is pharmaceutically acceptable and that possesses the desired pharmacological activity of the parent compound. Such salts include, for example, acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclol2.2.21-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. These may include cations based on the alkali and alkaline earth metals, such as sodium, lithium, potassium, calcium, magnesium, and the like, as well as non-toxic ammonium, tetramethylammonium, tetramethylammonium, methylamine, dimethylamine, trimethylamine, triethylamine, ethylamine, and the like.

The phrase “pharmaceutically acceptable” means that which is useful in preparing a pharmaceutical composition that is generally safe, non-toxic, and is not biologically or otherwise undesirable and is acceptable for human pharmaceutical use.

Furthermore, in addition to the above compounds, active pharmaceutical ingredients also include solvates of any of the above-mentioned compounds. The term “solvate” refers to an aggregate that comprises one or more molecules of active pharmaceutical ingredient with one or more molecules of a solvent. The solvent may be water, in which case the solvate may be a hydrate. Alternatively, the solvent may be an organic solvent. In one embodiment, “solvate” refers to the active pharmaceutical ingredient in its state prior to dissolution. Alternatively, the solid particles of a suspended active pharmaceutical ingredient may comprise a co-precipitated solvent.

In certain embodiments, the fill composition may also include nutraceuticals, such as vitamins, minerals, or supplements in addition to an active pharmaceutical ingredient or instead of an active pharmaceutical ingredient. It should be understood that any reference to API throughout the description (e.g., concentration) may also be suitable for a different active agent, such as a nutraceutical (i.e., vitamin, mineral, and/or supplement).

In some embodiments, the lipids in the dosage form may be selected, without limitation, from the group consisting of almond oil, argan oil, avocado oil, borage seed oil, canola oil, cashew oil, castor oil, hydrogenated castor oil, cocoa butter, coconut oil, colza oil, corn oil, cottonseed oil, grape seed oil, hazelnut oil, hemp oil, hydroxylated lecithin, lecithin, linseed oil, macadamia oil, mango butter, manila oil, mongongo nut oil, olive oil, palm kernel oil, palm oil, peanut oil, pecan oil, perilla oil, pine nut oil, pistachio oil, poppy seed oil, pumpkin seed oil, peppermint oil, rice bran oil, safflower oil, sesame oil, shea butter, soybean oil, sunflower oil, hydrogenated vegetable oil, walnut oil, and watermelon seed oil. Other oil and fats may include, but not be limited to, fish oil (omega-3), krill oil, garlic oil, animal or vegetable fats, e.g., in their hydrogenated form, free fatty acids and mono-, di-, and tri-glycerides with C8-, C10-, C12-, C14-, C16-, C18-, C20- and C22-fatty acids, fatty acid esters like EPA and DHA Sand combinations thereof.

According to certain embodiments, active agents may include lipid-lowering agents including, but not limited to, statins (e.g., lovastatin, simvastatin, pravastatin, fluvastatin, atorvastatin, rosuvastatin, and pitavastatin), fibrates (e.g, clofibrate, ciprofibrate, bezafibrate, fenofibrate, and gemfibrozil), niacin, bile acid sequestrants, ezetimibe, lomitapide, phytosterols, and the pharmaceutically acceptable salts, hydrates, solvates and prodrugs thereof, mixtures of any of the foregoing, and the like.

Suitable nutraceutical active agents may include, but are not limited to, 5-hydroxytryptophan, acetyl L-carnitine, alpha lipoic acid, alpha-ketoglutarates, bee products, betaine hydrochloride, bovine cartilage, caffeine, cetyl myristoleate, charcoal, chitosan, choline, chondroitin sulfate, coenzyme Q10, collagen, colostrum, creatine, cyanocobalamin (Vitamin 812), dimethylaminoethanol, fumaric acid, germanium sequioxide, glandular products, glucosamine HCl, glucosamine sulfate, hydroxyl methyl butyrate, immunoglobulin, lactic acid, L-Carnitine, liver products, malic acid, maltose-anhydrous, mannose (d-mannose), methyl sulfonyl methane, phytosterols, picolinic acid, pyruvate, red yeast extract, S-adenosylmethionine, selenium yeast, shark cartilage, theobromine, vanadyl sulfate, and yeast.

Suitable nutritional supplement active agents may include vitamins, minerals, fiber, fatty acids, amino acids, herbal supplements or a combination thereof.

Suitable vitamin active agents may include, but are not limited to, the following: ascorbic acid (Vitamin C), B vitamins, biotin, fat soluble vitamins, folic acid, hydroxycitric acid, inositol, mineral ascorbates, mixed tocopherols, niacin (Vitamin B3), orotic acid, para-aminobenzoic acid, panthothenates, panthothenic acid (Vitamin B5), pyridoxine hydrochloride (Vitamin B6), riboflavin (Vitamin B2), synthetic vitamins, thiamine (Vitamin B1), tocotrienols, vitamin A, vitamin D, vitamin E, vitamin F, vitamin K, vitamin oils and oil soluble vitamins

Suitable herbal supplement active agents may include, but are not limited to, the following: arnica, bilberry, black cohosh, cat's claw, chamomile, echinacea, evening primrose oil, fenugreek, flaxseed, feverfew, garlic oil, ginger root, ginko biloba, ginseng, goldenrod, hawthorn, kava-kava, licorice, milk thistle, psyllium, rauowolfia, senna, soybean, St. John's wort, saw palmetto, turmeric, valerian.

Minerals active agents may include, but are not limited to, the following: boron, calcium, chelated minerals, chloride, chromium, coated minerals, cobalt, copper, dolomite, iodine, iron, magnesium, manganese, mineral premixes, mineral products, molybdenum, phosphorus, potassium, selenium, sodium, vanadium, malic acid, pyruvate, zinc and other minerals.

Examples of other possible active agents include, but are not limited to, antihistamines (e.g., ranitidine, dimenhydrinate, diphenhydramine, chlorpheniramine and dexchlorpheniramine maleate), non-steroidal anti-inflammatory agents (e.g., aspirin, celecoxib, Cox-2 inhibitors, diclofenac, benoxaprofen, flurbiprofen, fenoprofen, flubufen, indoprofen, piroprofen, carprofen, oxaprozin, pramoprofen, muroprofen, trioxaprofen, suprofen, aminoprofen, fluprofen, bucloxic acid, indomethacin, sulindac, zomepirac, tiopinac, zidometacin, acemetacin, fentiazac, clidanac, oxpinac, meclofenamic acid, flufenamic acid, niflumic acid, tolfenamic acid, diflurisal, flufenisal, piroxicam, sudoxicam, isoxicam, aceclofenac, aloxiprin, azapropazone, benorilate, bromfenac, carprofen, choline magnesium salicylate, diflunisal, etodolac, etoricoxib, faislamine, fenbufen, fenoprofen, flurbiprofen, ibuprofen, indometacin, ketoprofen, ketorolac, lornoxicam, loxoprofen, meloxicam, mefenamic acid, metamizole, methyl salicylate, magnesium salicylate, nabumetone, naproxen, nimesulide, oxyphenbutazone, parecoxib, phenylbutazone, salicyl salicylate, sulindac, sulfinpyrazone, tenoxicam, tiaprofenic acid, tolmetin. pharmaceutically acceptable salts thereof and mixtures thereof) and acetaminophen, anti-emetics (e.g., metoclopramide, methylnaltrexone), anti-epileptics (e.g., phenyloin, meprobmate and nitrazepam), vasodilators (e.g., nifedipine, papaverine, diltiazem and nicardipine), anti-tussive agents and expectorants (e.g. codeine phosphate), anti-asthmatics (e.g. theophylline), antacids, anti-spasmodics (e.g. atropine, scopolamine), antidiabetics (e.g., insulin), diuretics (e.g., ethacrynic acid, bendrofluthiazide), anti-hypotensives (e.g., propranolol, clonidine), antihypertensives (e.g., clonidine, methyldopa), bronchodilatiors (e.g., albuterol), steroids (e.g., hydrocortisone, triamcinolone, prednisone), antibiotics (e.g., tetracycline), antihemorrhoidals, hypnotics, psychotropics, antidiarrheals, mucolytics, sedatives, decongestants (e.g. pseudoephedrine), laxatives, vitamins, stimulants (including appetite suppressants such as phenylpropanolamine) and cannabinoids, as well as pharmaceutically acceptable salts, hydrates, solvates, and prodrugs thereof.

The active agent may also be a benzodiazepine, barbiturate, stimulant, or mixtures thereof. The term “benzodiazepine” refers to a benzodiazepine and drugs that are derivatives of a benzodiazepine that are able to depress the central nervous system. Benzodiazepines include, but are not limited to, alprazolam, bromazepam, chlordiazepoxide, clorazepate, diazepam, estazolam, flurazepam, halazepam, ketazolam, lorazepam, nitrazepam, oxazepam, prazepam, quazepam, temazepam, triazolam, as well as pharmaceutically acceptable salts, hydrates, solvates, prodrugs and mixtures thereof. Benzodiazepine antagonists that can be used as active agent include, but are not limited to, flumazenil as well as pharmaceutically acceptable salts, hydrates, solvates and mixtures thereof.

The term “barbiturate” refers to sedative-hypnotic drugs derived from barbituric acid (2, 4, 6,-trioxohexahydropyrimidine). Barbiturates include, but are not limited to, amobarbital, aprobarbotal, butabarbital, butalbital, methohexital, mephobarbital, metharbital, pentobarbital, phenobarbital, secobarbital as well as pharmaceutically acceptable salts, hydrates, solvates, prodrugs, and mixtures thereof. Barbiturate antagonists that can be used as active agent include, but are not limited to, amphetamines as well as pharmaceutically acceptable salts, hydrates, solvates and mixtures thereof.

The term “stimulant” includes, but is not limited to, amphetamines such as dextro amphetamine resin complex, dextroamphetamine, methamphetamine, methylphenidate, as well as pharmaceutically acceptable salts, hydrates, and solvates and mixtures thereof. Stimulant antagonists that can be used as active agent include, but are not limited to, benzodiazepines, as well as pharmaceutically acceptable salts, hydrates, solvates and mixtures thereof.

The present invention is suitable for delivery of abuse-susceptible active pharmaceutical ingredients since the fill composition can provide a degree of abuse deterrence by, for example, making it difficult to isolate and purify the active pharmaceutical ingredient from the fill composition. The fill composition of the present invention is also suitable for controlled release delivery of the API, as well as for high potency API's which are preferably released into the subject in relatively small amounts over an extended time period (such as from about 2 hours to about 24 hours).

The API is preferably present in the controlled release fill composition in an amount of from about 5 wt. % to about 60 wt. %, based on the total weight of the controlled release fill composition. More preferably, the API is present in the controlled release fill composition in an amount of from about 10 wt. % to about 30 wt. %, based on the total weight of the controlled release fill composition.

In certain embodiments, the API (or active agent) is present in the controlled release fill composition in an amount of at least about 1 wt. %, at least about 5 wt. %, at least about 10 wt. %, at least about 15 wt. %, at least about 20 wt. %, at least about 25 wt. %, or at least about 30 wt. % and up to about 35 wt. %, up to about 40 wt. %, up to about 45 wt. %, up to about 50 wt. %, up to about 55 wt. %, or up to about 60 wt. %, based on a total weight of the controlled release fill composition. In certain embodiments, the API (or active agent) is present in the controlled release fill composition in an amount of from about 12 wt. % to about 18 wt. %, from about 19 wt. % to about 25 wt. %, from about 24 wt. % to about 32 wt. %, from about 4 wt. % to about 10 wt. %, or from about 25 wt. % to about 42 wt. %, based on the total weight of the controlled release fill composition. The concentration ranges of the active agent described herein may refer to a concentration of a single API (regardless of the number of APIs in the fill composition) or to the cumulative concentration of all APIs in the fill composition (if more than one API is present in the fill composition). Similarly, the concentrations of the API(s) may be applicable to active agents that are not pharmaceutical ingredients, such as, without limitation, nutraceuticals and other active agents as described above.

The polyethylene oxide (PEO) in the controlled release fill composition has a number average molecule weight from about 0.05 million daltons to about 15 million daltons, more preferably from about 500,000 daltons to about 10,000,000 daltons, and most preferably from about 1,000,000 daltons to about 8,000,000 daltons. In embodiments, PEOs that may be utilized have a number average molecular weight that ranges from any one of about 0.05M, about 0.5M Dalton, about 1M Dalton, about 2M Dalton, about 3M Dalton, or about 4M Dalton to any of about 5M about, 7M Dalton, about 10M Dalton, about 12M Dalton, about 15M Dalton, or about 20M Dalton, or any sub-range or single value therein. In one embodiment, the number average molecular weight of the polyethylene oxide in the controlled release fill composition ranges from about 0.05M Dalton to about 15M Dalton. In one embodiment, the number average molecular weight of the polyethylene oxide in the controlled release fill composition ranges from about 1M Dalton to about 10M Dalton. In one embodiment, the number average molecular weight of the polyethylene oxide in the controlled release fill composition ranges from about 1M Dalton to about 8M Dalton. In one embodiment, the number average molecular weight of the polyethylene oxide in the controlled release fill composition ranges from about 2M Dalton to about 5M Dalton.

The PEO is employed in the controlled release fill composition in an amount of at least 21.5 wt. %, based on the total weight of the controlled release fill composition. In an alternative embodiment, the PEO is in present in the controlled release fill composition in an amount from about 10 wt. % to about 65 wt. %, based on the total weight of the controlled release fill composition. Most preferably the PEO is present in the controlled release fill composition in an amount of about 25 wt. % to about 40 wt. %, based on the total weight of the controlled release fill composition.

In embodiments, the PEO is present in the controlled release fill composition in an amount of at least about 8 wt. %, at least about 10 wt. %, at least about 12 wt. %, at least about 14 wt. %, at least about 16 wt. %, at least about 18 wt. %, or at least about 20 wt. % up to about 25 wt. %, up to about 35 wt. %, up to about 45 wt. %, up to about 55 wt. %, or up to about 65 wt. %, or any sub-range therein, based on a total weight of the controlled release fill composition. In certain embodiments, the controlled release fill composition includes from about 8 wt. % to about 15 wt. %, from about 16 wt. % to about 20 wt. %, from about 22 wt. % to about 28 wt. %, from about 15 wt. % to about 30 wt. %, from about 20 wt. % to about 42 wt. %, from about 10 wt. % to about 35 wt. %, or from about 11 wt. % to about 40.5 wt. % PEO, based on the total weight of the controlled release fill composition.

In an alternative embodiment, the PEO may be present in the controlled release fill composition in any suitable amount when the water and/or hydrophilic carrier is present in an amount of up to 65 wt. %, based on the total weight of the controlled release fill composition. In this embodiment, the minimum amount of water and/or hydrophilic carrier may optionally be at least about 30 wt. %, or at least about 40 wt. %, or at least about 55 wt. %, based on the total weight of the controlled release fill composition. In these alternative embodiments, the amount of PEO in the controlled release fill composition can be from about 5 wt. % to about 35 wt. %, or about 20 wt. %, based on the total weight of the controlled release fill composition.

In certain embodiments, the PEO and the water and/or hydrophilic carrier may be present in the controlled release fill composition in any suitable amount such that the weight ratio of the PEO to the water and/or the hydrophilic carrier (individually or cumulatively) ranges from about 10:1 to about 1:10, from about 8:1 to about 1:8, from about 5:1 to about 1:5, from about 3:1 to about 1:3, from about 2:1 to about 1:2, from about 10:1 up to 1:3, from about 8:1 up to 1:3, from about 5:1 up to 1:3, from about 3:1 up to 1:3, from about 2:1 up to 1:3, from about 1:1 up to 1:3, from about 10:1 to about 1:2, from about 8:1 to about 1:2, from about 5:1 to about 1:2, from about 3:1 to about 1:2, from about 1:1 to about 1:2, or any sub-range or single weight ratio value therein. In one embodiment, the weight ratio of the PEO to the water and/or the hydrophilic carrier (individually or cumulatively) ranges from about 2:1 to about 1:2. In one embodiment, the weight ratio of the PEO to the water and/or the hydrophilic carrier (individually or cumulatively) ranges from about 3:1 up to 1:3.

Suitable polyethylene oxides are typically non-ionic, high molecular weight, water-soluble polyethylene oxide resins. Exemplary PEO resins of this type are the Polyox™ water-soluble resins available from DuPont Pharma Solutions. These PEO resins are typically used as thickeners and rheology control agents. In the present invention, these water-soluble PEO resins can be employed to modify or control the release of the API from a softgel capsule and/or hard capsule and/or a capsule fill composition. PEO resins may also be employed in the fill compositions to deter abuse of the API that is contained in the fill compositions.

The ratio of the PEO to other components of the fill composition (such as the API or other controlled release materials, if present) may be adjusted to attain a target release profile for the API. In certain embodiments, the wt:wt ratio of the PEO to the API may range from about 10:1 to about 1:10, about 8:1 to about 1:8, about 5:1 to about 1:5, about 3:1 to about 1:3, or about 1:1.

In certain embodiments, the hydrophilic carrier in the fill composition has a number average molecule weight of from about 50 daltons to about 7000 daltons, from about 200 daltons to 5000 daltons, more preferably, the number average molecular weight of the hydrophilic carrier is from about 300 daltons to about 3000 daltons, and most preferably the number average molecule weight of the hydrophilic carrier is from about 400 daltons to about 1500 daltons. In certain embodiments, the hydrophilic carrier may include compounds with a number average molecular weight that is below 200 daltons.

Examples of suitable hydrophilic carriers are hydrophilic solvents that include polyoxyethylene derivatives of a sorbitan ester, such as sorbitan monolaurate (Polysorbate 20), Polysorbate 80, Polysorbate 60, polyoxyethylene 20 sorbitan trioleate (Polysorbate 85), and other hydrophilic carriers including polyethylene glycol, polypropylene glycol, propylene glycol, acetic acid, formic acid, other hydrophilic surfactants and mixtures thereof.

The hydrophilic carrier is preferably selected from polyethylene glycol and polypropylene glycol. In addition, or as an alternative to these hydrophilic carriers, water may also be added to the fill compositions described herein. Most preferably, the hydrophilic carrier is polyethylene glycol. The polyethylene glycol will typically have number average molecular weight of from 300 to 7000 g/mol. The term “high molecular weight polyethylene glycol,” as used herein, refers to polyethylene glycol with a number average molecular weight higher than 1500 daltons, e.g., 1500 daltons to 7000 daltons. Combinations of two or more polyethylene glycols having different molecular weights may also be employed. Polypropylene glycol is a preferred additional component of the hydrophilic carrier when a viscosity reduction in the liquid fill composition is required.

In one embodiment, the water and/or hydrophilic carrier is included in the controlled release fill composition in an amount of up to 65 wt. %, based on the total weight of the controlled release fill composition. In another embodiment, the water and/or hydrophilic carrier is included in the controlled release fill composition in an amount of from about 10 wt. % to about 75 wt. %, or 30 wt. % to about 70 wt. %, based on the total weight of the controlled release fill composition. Preferably, the water and/or hydrophilic carrier is included in the controlled release fill composition in an amount of from about 40 wt. % to about 60 wt. %, based on the total weight of the controlled release fill composition.

In certain embodiments, the water and/or hydrophilic carrier is included in the controlled release fill composition in an amount of above 0 wt. %, at least about 15 wt. %, or at least about 30 wt. % up to about 45 wt. %, up to about 60 wt. %, up to about 70 wt. %, or up to about 80 wt. %, based on a total weight of the controlled release fill composition. In certain embodiments, the controlled release fill composition includes from about 5 wt. % to about 15 wt. %, from about 15 wt. % to about 28 wt. %, from about 20 wt. % to about 32 wt. %, from about 20 wt. % to about 42 wt. %, from about 22 wt. % to about 45 wt. %, from about 40 wt. % to about 45 wt. %, from about 40 wt. % to about 55 wt. %, from about 35 wt. % to about 55 wt. %, from about 56 wt. % to about 77 wt. %, from about 40 wt. % to about 79 wt. %, or from about 29 wt. % to about 66 wt. % water and/or hydrophilic carrier, based on the total weight of the controlled release fill composition. The concentration ranges of the hydrophilic carrier described herein may refer to a concentration of a single hydrophilic carrier material (regardless of the number of hydrophilic carrier materials in the fill composition) or to the cumulative concentration of all hydrophilic carrier materials in the fill composition (if more than one hydrophilic carrier material is present in the fill composition).

In another embodiment, the hydrophilic carrier can be present in the controlled release fill composition in any amount so long at the polyethylene oxide is present in an amount of at least 21.5 wt. % of the controlled release fill composition, based on the total weight of the controlled release fill composition. In this embodiment, the hydrophilic carrier is typically present in amounts of up to 65 wt. %, or from 10 wt. % to 65 wt. %, or from 30 wt. % to 60 wt. %, or from 30 wt. % to 55 wt. %, based on the total weight of the controlled release fill composition. The hydrophilic carrier is used to dissolve, disperse and/or suspend the other components of the liquid fill composition in a liquid and may also function to adjust the viscosity of the liquid fill composition to a desired viscosity for the encapsulation step.

The liquid fill composition may have a viscosity in the range of 1000 cP to 100,000 cP, or from 5,000 cP to 80,000 cP, or from 10,000 cP to 60,000 cP at the time of filling (or encapsulation within) the capsule. The viscosity of the liquid fill composition was determined at 20° C. using a HAAKE RheoStress 600 rheometer equipped with a 40 mm flat plate geometry. The geometry oscillated at 1 Hz with a gap setting of 2 mm

The fill compositions described herein provide the ability to control the release of the API from the dosage form. The PEO amount and/or molecular weight of the PEO component can be adjusted to optimize the release rate of the API from the capsule.

A significant advantage of the fill composition being liquid during processing, is that it obviates the need to handle powders in the process for making the dosage form, except in the initial mixing step, in contrast to tablet dosage forms which generally require handling of powders throughout the process of making the dosage form. Further, processing of the liquid fill compositions described herein can reduce or obviate the need to include flowability enhancers or processability enhancers to facilitate processing. Similarly, given that the fill compositions are liquid at ambient temperatures, there is no need to heat them prior to encapsulation, which heating could be harmful to heat sensitive materials such as those utilized in shell compositions of certain softgel capsules. The ability to provide a liquid fill for encapsulation allows for use of softgel and hard-shell capsules to provide controlled release dosage forms.

Another embodiment relates to capsules containing the above-described fill compositions. These capsules may be softgel capsules, soft capsules or hard capsules. In the case of soft capsules, any size capsule may be employed. In one embodiment, a softgel gelatin capsule encapsulates any of fill compositions described herein.

The dry shell accounts for about 30 wt. % to about 60 wt. %, based on the total weight of the filled soft capsule. In this case, the controlled release fill composition accounts for about 40 wt. % to about 70 wt. %, based on the total weight of the filled soft capsule.

For hard capsules, the capsule shell accounts for up to about 10 wt. %, based on the total weight of the filled hard capsule. In this case, the controlled release fill composition accounts for up to about 90 wt. %, based on the total weight of the filled hard capsule. The hard capsules will be sealed using conventional hard capsule sealing methods known in the art to prevent leakage of the liquid fill composition from the capsule during encapsulation.

The softgel capsules may contain gelatin but need not be gelatin-based capsules. Other suitable, conventional softgel capsules may also be employed. An advantage of non-gelatin soft capsules is that higher encapsulation temperatures of up to 70° C. can be employed in the encapsulation step to ensure that the fill composition is sufficiently flowable which allows use of high viscosity fills such as those containing, for example, high molecular weight hydrophilic excipients.

Hard shell capsules provide a similar flexibility in the encapsulation step since hard shell capsules also allow for use of such higher encapsulation temperatures of up to 70° C.

When non-gelatin soft capsules or hard capsules that tolerate heating above the melting point of the PEO (about 50° C.), the liquid fill can be heated to above the melting point of the PEO after encapsulation to melt the PEO and form the desired substantially homogeneous controlled release fill composition by cooling and solidification of the melted fill composition. As a result of this melting step, a more uniform fill composition is formed in situ within the capsule. This uniformity of the fill composition is promoted by the presence of the hydrophilic carrier which can also function as a plasticizer during this melting step.

A significant advantage of the use of polyethylene oxide as the primary rate controlling component of the liquid fill composition is that it does not tend to be as tacky or sticky as other rate-controlling polymers thereby facilitating the encapsulation process and ensuring a more homogeneous fill composition. While other additional rate-controlling polymers can be employed, the amounts of such rate-controlling polymers must be carefully selected to prevent this stickiness or tackiness from causing problems during the encapsulation process that may lead to an inferior product.

Preferably, the API release rate from the controlled release fill composition is such that less than 80% of the API is released after 0.5 hours in a fiberoptic dissolution test using USP Apparatus II using a paddle speed of 100 RPM at 37° C. in 500 ml of biological, artificial, or simulated gastric fluid, such as 0.1 N HCl and/or biological, artificial, or simulated intestinal fluid, such as pH 6.8 phosphate buffer and/or water. More preferably, the API release rate from the controlled release capsule is such that less than 80% of the API is released after 1 hour in a fiberoptic dissolution test using USP Apparatus II using a paddle speed of 100 RPM at 37° C. in 500 ml of biological, artificial, or simulated gastric fluid, such as 0.1 N HCl and/or biological, artificial, or simulated intestinal fluid, such as pH 6.8 phosphate buffer and/or water. The fill composition is the rate controlling composition independent from the capsule shell, whether softgel or hard. In certain embodiments, the controlled release fill composition releases about 10 wt. % to about 30 wt. % of the API at 1 hour, about 15 wt. % to about 50 wt. % of API at 2 hours, about 20 wt. % to about 80 wt. % of API at 4 hours, about 40 wt. % to about 95 wt. % of API at 8 hours, from about 65 wt. % to about 100 wt. % of the API at 12 hours, and greater than 90 wt. % of API at 24 hours, in each case, measured in vitro in a fiber optic dissolution test using USP Apparatus II (paddle) at 100 RPM at 37° C. in 500 ml of biological, artificial, or simulated gastric fluid, such as 0.1 N HCl and/or biological, artificial, or simulated intestinal fluid, such as pH 6.8 phosphate buffer and/or water.

The fill composition may comprise one or more optional ingredients including a surfactant(s), plasticizer(s), and one or more API release rate controlling polymers other than PEO. The optional additional API release rate controlling polymers that can be included in the fill composition are preferably selected from one or more of cellulose derivative (e.g., microcrystalline cellulose, sodium carboxymethyl cellulose, methylcellulose, ethyl cellulose, hydroxyethyl cellulose, hydroxypropyl cellulose, hydroxypropyl methylcellulose, or combinations thereof), chitosan, carnauba wax, carbomers, polysaccharides, gums (e.g., acacia, pectin, agar, tragacanth, guar gum, xanthan gum, locust bean gum, tara gum, karaya, gellan gum, welan gum, and rhamsan gum, or combinations thereof), or combinations thereof.

Examples of optional surfactants include polyoxyl 40 hydrogenated castor oil, caprylocaproyl macrogol-8 glyceride, glycerol, macrogolglycerol hydroxystearate, Cremophor® RH 40, macrogolglycerol ricinoleate, Cremophor® EL, glycerolmonooleate 40, Peceol™, macrogolglycerol linoleate, Labrafil M 2125 CS, propylene glycol monolaurate FCC, Lauroglycol FCC, polyglycerol-6-dioleate, polyglycerol-3-dioleate, Plurol® Oleique, propylene glycol monocaprylate, Capryol® 90, sorbitan monolaurate, Span® 20, sorbitan monooleate, Span® 80, Vitamin E-polyethylenglycol-succinate, Labrasol®, macrogol-32-glycerol-laurate, Gelucire 44/14, glycerylmonocaprate/caprylate, Capmul MCM and mixtures thereof.

Optional additional API release controlling polymers may include cellulose derivatives such as such as methylcellulose, ethylcellulose, hydroxyethyl cellulose, hydroxypropyl cellulose, and carboxymethyl cellulose, biological gums and other gelling agents. Biological gums may be selected from acacia, pectin, agar, tragacanth, guar gum, xanthan gum, locust bean gum, tara gum, karaya, gellan gum, welan gum, and rhamsan gum, Other gelling agents may include pectin, starch, carbomer, sodium alginate, gelatin, casein, carrageenans, collagen, dextran, succinoglucon and polyvinyl alcohol clays.

Another embodiment relates to a method of producing a controlled release softgel capsule containing a controlled release fill composition containing a polyethylene oxide resin. This process is designed to accommodate softgel capsules which are not compatible with high encapsulation temperatures due to the relatively low melting points of the capsule shell material. For example, gelatin-based softgels may begin to melt at temperatures of from 33-45° C., depending to some extent on the water content of the capsule shell material at the time of encapsulation. For such lower melting temperature capsule shell materials, a method has been devised to fill the capsules with a liquid fill composition at lower temperatures. A significant advantage of this method is that it can be used to ultimately provide an encapsulated highly viscous liquid, semi-solid or solid fill composition. In this method, a solid solution or semi-solid fill is formed in situ inside the capsule as a result of the heating step carried out after encapsulation.

In this method, suspensions and dispersions, rather than a solution, can be employed. A softgel capsule shell will typically contain water in amounts of up to 20 wt. %, based on the total weight of the capsule shell, upon completion of the encapsulation step. During the encapsulation and a subsequent drying step, a significant portion, i.e. up to about 70%, of the water in the capsule shell will migrate into the fill composition and solubilize solid components within the suspension/dispersion of the fill composition, like PEO, in situ to form the desired solution. With this method, solubilization of solid components within the fill composition (e.g., PEO) occurs in situ. The water content in the fill composition, prior to encapsulation, is sufficiently low to limit or avoid solubilization of at least some of the constituents of the fill composition (such as PEO) prior to the encapsulation and drying steps. Premature solubilization of certain constituents within the fill composition (i.e., before encapsulation and drying), could increase the viscosity of the fill composition and hinder processability. Typically, the initial fill composition will have a water content of about 2 wt. % to about 10 wt. %, based on the total weight of the fill composition, to avoid premature solubilization of the PEO component of the fill composition prior to encapsulation. After encapsulation of the fill composition, a portion of the water from the softgel capsule shell migrates into the fill composition, typically raising the water content of the fill composition to from about 15 wt. % to about 20 wt. %, based on the total weight of the encapsulated fill composition, thereby causing solubilization of the PEO in the encapsulated fill composition. During the subsequent drying water is gradually removed until the water content of the encapsulated fill composition falls below 10 wt. %, based on the total weight of the encapsulated and dried fill composition. After the final heating step (also referred to as an annealing step), the water content of the final encapsulated fill composition is further reduced to from about 5 wt. % to about 8 wt. %, based on the total weight of the final encapsulated fill composition. The final encapsulated fill composition forms a solid solution of PEO in the hydrophilic carrier.

This process of forming the solid solution in situ is important since it provides a more uniform distribution of the API in the fill composition, unlike powder filled capsules or other solid dosage forms. Uniform distribution of the API is an important characteristic for delivery of high potency and/or low dose API's since such API's should be delivered at a relatively constant rate over time to avoid over or under dosing. In certain embodiments, the uniform distribution of the API in the fill composition enables zero order release of the API from the controlled release fill composition (where the API is delivered at a relatively constant rate over time, e.g., from about 2 hours to about 12 hours, or from about 2 hours to about 24 hours).

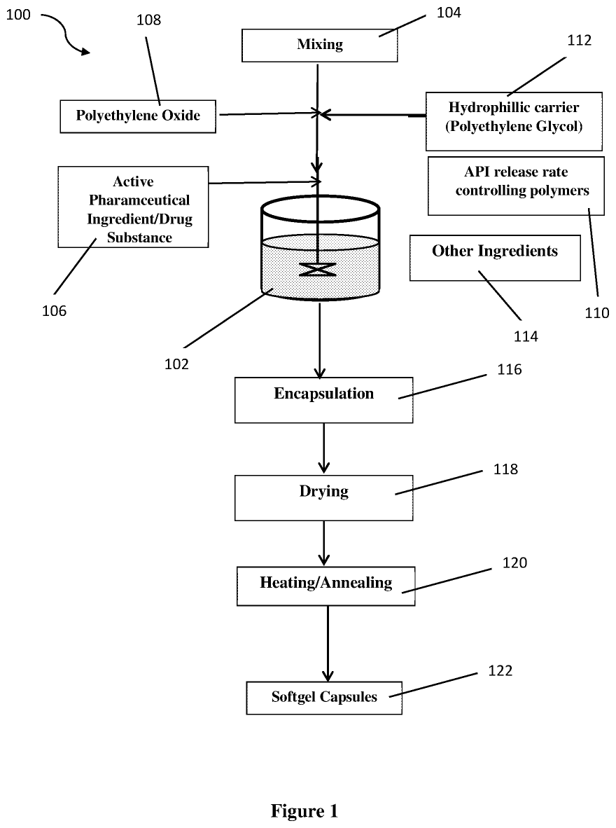

FIG. 1 shows a flow diagram 100 of the steps and materials used in this method to manufacture capsules. In this method, the fill composition 102 is mixed in mixing step 104 using any suitable apparatus known in the art to be capable for mixing the fill composition 102. The fill composition 102 includes at least an active pharmaceutical ingredient (API) 106, polyethylene oxide 108, and, optionally one or more additional API release rate controlling polymers 110, and water and/or a hydrophilic carrier 112. The fill composition 102 may also include other additional ingredients 114 (e.g., pharmaceutically acceptable excipients), such as inactive ingredients and other suitable components such as surfactant(s) and plasticizer(s) for use in fill compositions that are known in the art.

The API 106 can be a pharmaceutical component that can be a single ingredient or a mixture of one or more APIs as is known in the art. Preferably, the API 106 is selected from APIs classified in one of Biopharmaceutics Classification System Classes I, II, III, or IV. In certain embodiments, a nutraceutical, such as vitamins, minerals, or supplements are included instead of API 106 or in addition to API 106. In one embodiment, the API is a drug that is not susceptible to abuse. The API 106 is preferably mixed into the fill composition 102 in an amount from about 5 wt. % to about 60 wt. %, based on the total weight of the fill composition 102. More preferably, the API 106 is mixed into the fill composition 102 in an amount of from about 5 wt. % to about 40 wt. %, or from about 10 wt. % to about 30 wt. %, based on the total weight of the fill composition 102.

The polyethylene oxide 108 may have a number average molecular weight of from about 0.05 M daltons to about 15 M daltons, more preferably from about 0.5 M daltons to about 10 M daltons and most preferably from about 1,000,000 daltons to about 8,000,000 daltons. In one embodiment of the method, the polyethylene oxide 108 is mixed into the fill composition 102 in an amount of at least 21.5 wt. %, based on the total weight of the fill composition 102. In another embodiment, the polyethylene oxide 108 is mixed into the fill composition 102 in an amount from about 10 wt. % to about 65 wt. %, based on the total weight of the fill composition 102, and most preferably the polyethylene oxide 108 is mixed into the fill composition 102 in an amount of about 25 wt. % to about 40 wt. %, based on the total weight of the fill composition 102.

In another embodiment of the method, the polyethylene oxide 108 can be mixed into the fill composition 102 in any amount so long as the hydrophilic carrier 112 is present in an amount of up to 65 wt. %, based on the total weight of the fill composition 102. In this embodiment, the minimum amount of hydrophilic carrier 112 may optionally be at least 55 wt. %, based on the total weight of the fill composition 102. In this embodiment, the minimum amount of hydrophilic carrier 112 may optionally be at least about 30 wt. %, or at least about 40 wt. %, or at least about 55 wt. %, based on the total weight of the fill composition 102. In this alternative embodiment, the amount of PEO 108 can be from about 5 wt. % to about 35 wt. %, or about 20 wt. %.

The hydrophilic carrier 112 mixed into the fill composition 102 may have a number average molecular weight from 50 daltons to 7000 daltons, from 200 daltons to 5000 daltons, more preferably, the number average molecular weight of the hydrophilic carrier 112 is from about 300 daltons to about 3000 daltons, and most preferably the number average molecule weight of the hydrophilic carrier 112 is from about 400 daltons to about 1500 daltons. In certain embodiments, the hydrophilic carrier 112 may have a number average molecular weight that is lower than 200 daltons.

The hydrophilic carrier 112 is preferably selected from polyethylene glycol, polypropylene glycol, or other known hydrophilic solvents. Most preferably, the water and/or hydrophilic carrier 112 is polyethylene glycol. The water and/or hydrophilic carrier 112 is mixed into the fill composition 102 in an amount up to 65 wt. %, based on the total weight of the fill composition 102. In an alternative embodiment, the water and/or hydrophilic carrier 112 is mixed into the fill composition 102 in an amount of from about 30 wt. % to about 70 wt. %, based on the total weight of the fill composition 102. Preferably the water and/or hydrophilic carrier 112 is mixed into the fill composition 102 in an amount from about 40 wt. % to about 60 wt. %, based on the total weight of the fill composition 102.

In yet another embodiment, the water and/or hydrophilic carrier 112 can be present in the fill composition 102 in any amount when the polyethylene oxide 108 is present in an amount of at least 21.5 wt. %, based on the total weight of the fill composition 102.

In certain embodiments, the polyethylene oxide 108 and the water and/or hydrophilic carrier 112 may be present in the fill composition 102 in any suitable amount such that the weight ratio of the PEO 108 to the water and/or the hydrophilic carrier 112 (individually or cumulatively) ranges from about 10:1 to about 1:10, from about 8:1 to about 1:8, from about 5:1 to about 1:5, from about 3:1 to about 1:3, from about 2:1 to about 1:2, from about 10:1 up to 1:3, from about 8:1 up to 1:3, from about 5:1 up to 1:3, from about 3:1 up to 1:3, from about 2:1 up to 1:3, from about 1:1 up to 1:3, from about 10:1 to about 1:2, from about 8:1 to about 1:2, from about 5:1 to about 1:2, from about 3:1 to about 1:2, from about 1:1 to about 1:2, or any sub-range or single weight ratio value therein. In one embodiment, the weight ratio of the PEO 108 to the water and/or the hydrophilic carrier 112 (individually or cumulatively), in the fill composition 102, ranges from about 2:1 to about 1:2. In one embodiment, the weight ratio of the PEO 108 to the water and/or the hydrophilic carrier 112 (individually or cumulatively), in the fill composition 102, ranges from about 3:1 up to 1:3.

The one or more additional API release rate controlling polymers 110 that may be mixed into the fill composition 102 can be selected from one or more of the following polymers, hydroxypropyl methylcellulose, cellulose derivative, chitosan, carnauba wax, carbomer, and polysaccharides, or any other release rate controlling polymers described hereinbefore, or a combination thereof. After the fill composition 102 is mixed (step 104), the fill composition 102 is encapsulated (step 116) in a capsule shell to produce a capsule. After encapsulation step 116, the softgel capsule is preferably dried (step 118), though this step is optional. In certain embodiments, the drying step 118, if present, should not remove too much water from the capsule shell since water in the capsule shell that migrates into the fill composition during the subsequent heating step functions as a solubilizing agent to solubilize the fill composition in situ.

The softgel capsule is then heated (step 120) to a temperature of from about 40° C. to about 80° C. for a period of from about 10 minutes to about 180 minutes. More preferably the softgel capsule is heated (step 120) to a temperature of from about 45° C. to about 70° C. Most preferably, the softgel capsule is heated (step 120) to a temperature of from about 50° C. to about 60° C. More preferably, the softgel capsule is heated (step 120) for a period of from about 20 minutes to 120 minutes, and most preferably for a period of from about 30 minutes to 90 minutes. After the softgel capsule is heated (step 120) the final capsule 122 is formed. The purpose of this heating step (which may also be referred to as annealing or curing) is to solubilize particles within the suspension or dispersion-type liquid fill by using water that migrates from the capsule shell to the fill composition during the heating step. As a result, the fill composition forms a homogeneous solution which, upon cooling, solidifies to form a solid or semi-solid homogeneous solution in the fill composition that, at least in part, provides the controlled release property. Typically, a water content of about 10 wt. % to 15 wt. % in the capsule shell (e.g., softgel capsule shell) at the start of the heating step is used to provide enough water migration to the fill composition to form the fill composition solution. If the water content of the capsule shell is too high after encapsulation, an optional drying step can be employed prior to the heating (or annealing) step to reach the desired water content for the softgel capsule shell.

In this method, the capsule is prepared by a process that includes a step of heating (step 120) the capsule 122 containing the fill composition. The API release profile, which is exhibited by capsule 122, can be tailored by selection of the molecular weight and/or concentration of the PEO 108 in the fill composition. In some embodiments, the API release rate is such that 10-80% of the API 106 is released after 0.5 hours in a fiberoptic dissolution test using USP Apparatus II using a paddle speed of 100 RPM at 37° C. in 500 ml of biological, artificial, or simulated gastric fluid, such as 0.1 N HCl and/or biological, artificial, or simulated intestinal fluid, such as pH 6.8 phosphate buffer and/or water. More preferably, the release rate is such that less than 20-100% of the API is released after 1 hour, or 30-100% of the API is released after 6 hours, or 50-100% of the API is released after 12 hours, or 70-100% of the API is released after 18 hours, or 80-100% of the API is released after 24 hours, all as determined in a fiberoptic dissolution test using USP Apparatus II using a paddle speed of 100 RPM at 37° C. in 500 ml of biological, artificial, or simulated gastric fluid, such as 0.1 N HCl and/or biological, artificial, or simulated intestinal fluid, such as pH 6.8 phosphate buffer and/or water.

In certain embodiments, this method provides for a capsule encapsulating a controlled release fill composition, which releases about 10 wt. % to about 30 wt. % of the API at 1 hour, about 15 wt. % to about 50 wt. % of API at 2 hours, about 20 wt. % to about 80 wt. % of API at 4 hours, about 40 wt. % to about 95 wt. % of API at 8 hours, from about 65 wt. % to about 100 wt. % of the API at 12 hours, and greater than 90 wt. % of API at 24 hours, in each case, as measured in vitro in a fiber optic dissolution test using USP Apparatus II (paddle) at 100 RPM at 37° C. in 500 ml of biological, artificial, or simulated gastric fluid, such as 0.1 N HCl and/or biological, artificial, or simulated intestinal fluid, such as pH 6.8 phosphate buffer and/or water.

The method of the present invention may include an optional step of drying (step 118) the capsule 122 prior to the heating step 120. The drying step 118 may be carried out at a temperature of 20-30° C. for a time period of 24-240 hours under mild temperature and humidity (20-35° C. and 10-50% or 20-40% or 30% relative humidity) conditions.

In certain embodiments, the instant disclosure is also directed to a method of treating a condition comprising, administering to a subject in need thereof any of the capsules described herein. The term “condition” or “conditions” refers to those medical conditions that can be treated or prevented by administration to a subject of an effective amount of an active pharmaceutical ingredient.

In certain embodiments, the instant disclosure is directed to a method for tuning the dissolution profile of a controlled release fill composition, the method comprising: adjusting at least one of i)-v) to attain a target dissolution profile of the API: i) number average molecular weight of a polyethylene oxide in the controlled release fill composition; ii) concentration of the a polyethylene oxide in the controlled release fill composition; iii) water or hydrophilic carrier content in the controlled release fill composition; iv) annealing temperature; v) annealing duration.

The following examples are illustrative, but not limiting, of the present disclosure. Other suitable modifications and adaptations of the variety of conditions and parameters normally encountered in the field, and which are obvious to those skilled in the art, are within the scope of the disclosure. The following examples illustrate the practice of the present disclosure in some of the preferred embodiments.

### EXAMPLES

### Examples 1-6 Dissolution Profiles of Fill Compositions

A 2×3 full factorial design of experiment with duplicates was utilized for the design of the six (6) fill compositions used in Samples 1-12 as set forth in Table 1 below. Each of the compositions was prepared twice to enable assessment of the composition variability. Diphenhydramine HCl was used as a model drug for the active pharmaceutical ingredient in the fill compositions. “PEG 400” is an abbreviation for polyethylene glycol having a number average molecular weight of 400, “PEO” is an abbreviation for polyethylene oxide, “M” stands for “million”, “HCl” is an abbreviation for hydrogen chloride and “Mn” is an abbreviation for number average molecular weight. All PEO's used in Examples 1-12 were non-ionic and water soluble and were Polyox™ products obtainable from DuPont Pharma Solutions.

TABLE 1
Fill Compositions

| | | | | | Diphenhydramine |
|---|---|---|---|---|---|
| | PEG 400 | PEO Polyox ™ | PEO | Water | HCl |
| Sample | (g) | Grade | (g) | (g) | (g) |
| 1 | 14.0 | Mn 5M Da | 4.0 | 2.0 | 2.0 |
| 2 | 14.0 | Mn 0.9M Da | 4.0 | 2.0 | 2.0 |
| 3 | 14.0 | Mn 0.1M Da | 4.0 | 2.0 | 2.0 |
| 4 | 14.0 | Mn 5M Da | 4.0 | 2.0 | 2.0 |
| 5 | 10.0 | Mn 5M Da | 8.0 | 2.0 | 2.0 |
| 6 | 10.0 | Mn 0.1M Da | 8.0 | 2.0 | 2.0 |
| 7 | 10.0 | Mn 5M Da | 8.0 | 2.0 | 2.0 |
| 8 | 14.0 | Mn 0.9M Da | 4.0 | 2.0 | 2.0 |
| 9 | 10.0 | Mn 0.1M Da | 8.0 | 2.0 | 2.0 |
| 10 | 10.0 | Mn 0.9M Da | 8.0 | 2.0 | 2.0 |
| 11 | 10.0 | Mn 0.9M Da | 8.0 | 2.0 | 2.0 |
| 12 | 14.0 | Mn 0.1M Da | 4.0 | 2.0 | 2.0 |

The diphenhydramine capsules of Samples 1-12 using the fill compositions set forth in Table 1 were prepared as follows. First, the fill compositions were made by solubilizing the diphenhydramine HCl (DHP) in 2 ml of water and mixing the PEG 400 with the PEO to form two components. The aqueous DPH solution was then added to the PEG/PEO mixture. Each Size 0 capsule was filled with 0.55 g of the fill composition to provide a dose of 50 mg diphenhydramine per capsule. The capsules were then annealed at 60° C. for one (1) hour in an oven.

The dissolution studies were carried out using the prefilled Size 0 gelatin hardshell capsules containing fill compositions by fiberoptic dissolution using USP Apparatus II with paddle speeds of 50 rpm and 100 rpm, at 37° C. in 500 ml water as the dissolution medium. The fill compositions used for the dissolution studies are shown in Table 2.

TABLE 2
Fill Compositions Used for Dissolution Studies

| Formu- | PEG 400 | PEO Polyox ™ | PEO | Diphenhydramine | Water |
|---|---|---|---|---|---|
| lation | (g) | Grade | (g) | (g) | (g) |
| 1 | 10.0 | Mn 5M Da | 8.0 | 2.0 | 2.0 |
| 2 | 14.0 | Mn 5M Da | 4.0 | 2.0 | 2.0 |
| 3 | 10.0 | Mn 0.9M Da | 8.0 | 2.0 | 2.0 |
| 4 | 14.0 | Mn 0.9M Da | 4.0 | 2.0 | 2.0 |
| 5 | 10.0 | Mn 0.1M Da | 8.0 | 2.0 | 2.0 |
| 6 | 14.0 | Mn 0.1M Da | 4.0 | 2.0 | 2.0 |

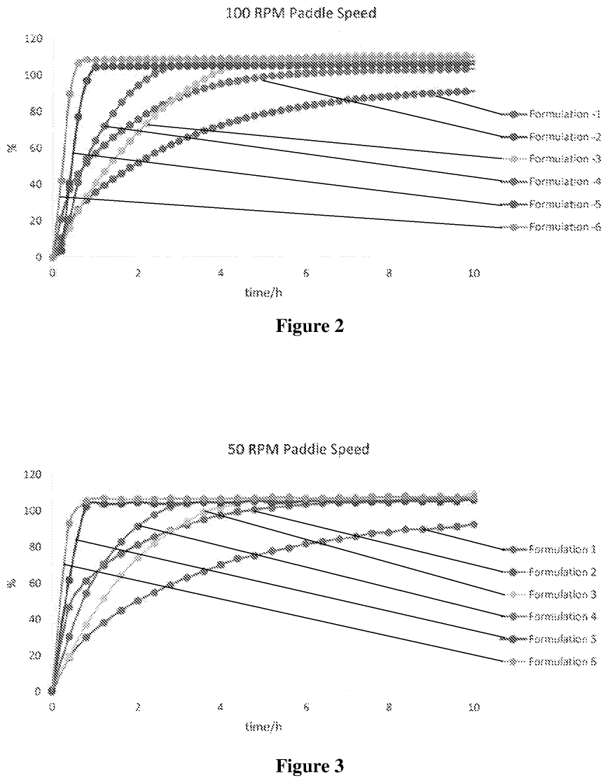

The dissolution profiles for the six (6) fill compositions listed in Table 2 at 100 RPM paddle speed are shown in FIG. 2. The dissolution profiles for the six (6) fill compositions listed in Table 2 at 50 RPM paddle speed are shown in FIG. 3.

The dissolution profiles were similar at 50 RPM and 100 RPM paddle speeds for each fill composition, indicating that the drug release mechanism was mainly by diffusion. The dissolution results show that higher molecular weight PEO and higher PEO concentration each resulted in a slower drug release. Fill compositions 5 and 6 prepared from 0.1M PEO had immediate release profiles while all the other fill compositions exhibited variable drug release rates as shown in FIGS. 2-3.

The Minitab 16 software package was used to analyze the collected dissolution data set. The time for the drug release to reach 90% was used as the dependent variable. The effect of PEO content and PEO molecular weight on the dependent variable was analyzed using the General Linear Model module in the Minitab 16 software package. The results are summarized in the following tables 3-4\6.

TABLE 3
General Linear Model: time to release
90% DHP v. PEO %, PEO Mn (MDa)

| Factor | Type | Levels | Values | | |
|---|---|---|---|---|---|
| PEO % | Fixed | 2 | 18.182 | 36.364 | |
| PEO Mn (MDa) | Fixed | 3 | 0.1 | 0.9 | 5.0 |

TABLE 4
Analysis of Variance for time 90% (h), using Adjusted SS for Tests

| Source | DF | Seq SS | Adj SS | Adj MS | F | P |
|---|---|---|---|---|---|---|
| PEO % | 1 | 29.482 | 29.482 | 29.482 | 22.14 | 0.000 |
| PEO Mn (MDa) | 2 | 116.323 | 116.323 | 58.162 | 43.68 | 0.000 |
| PEO %*PEO Mn (MDa) | 2 | 22.703 | 22.703 | 11.352 | 8.52 | 0.002 |
| Error | 18 | 23.970 | 23.970 | 1.332 | | |
| Total | 23 | 192.478 | | | | |

| | | |
|---|---|---|
| S = 1.15398 | R-Sq = 87.55% | R-Sq(adj) = 84.09% |

TABLE 5
Grouping Information Using Tukey Method and 95.0% Confidence

| PEO % | N | Mean | Grouping |
|---|---|---|---|
| 36.364 | 12 | 4.1500 | A |
| 18.182 | 12 | 1.9333 | B |

Means that do not share a letter are significantly different.

TABLE 6
Grouping Information Using Tukey Method and 95.0% Confidence

| PEO Mn (MDa) | N | Mean | Grouping |
|---|---|---|---|
| 5.0 | 8 | 5.9500 | A |
| 0.9 | 8 | 2.5500 | B |
| 0.1 | 8 | 0.6250 | C |

Means that do not share a letter are significantly different.

In the foregoing tables, the following abbreviations were employed:
DF—Degrees of Freedom
Seq SS—Sequential sums of squares which are measures of variation for different components of the model.
Adj SS—Adjusted sum of squares for a term is the increase in the regression sum of squares compared to a model with only the other terms
Adj MS—Adjusted mean squares measure how much variation a term or a model explains
F—F-value is the test statistic used to determine whether the model is missing higher-order terms that include the predictors in the current model.
P—Probability. P<0.05 indicates that the result is significant; otherwise, it is not significant.
N—Number of data points

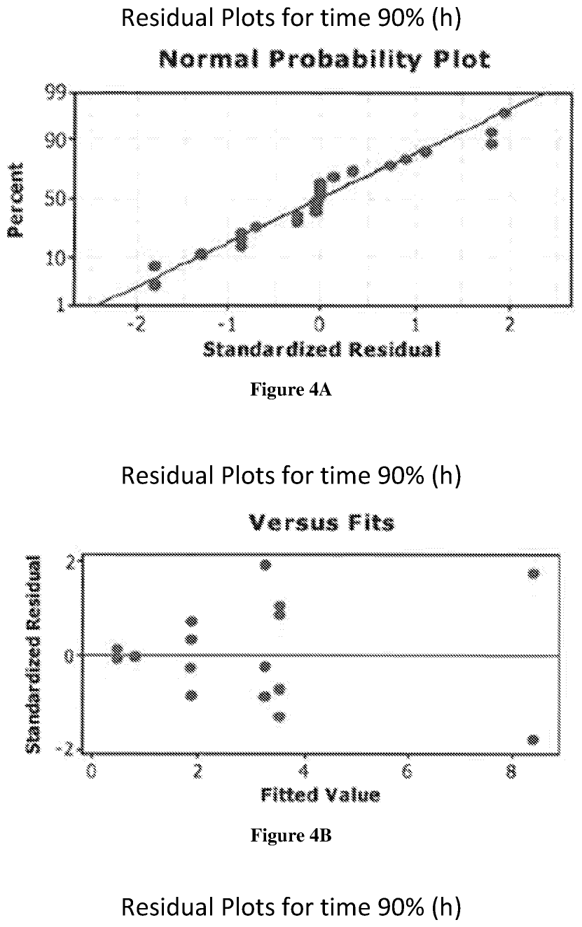

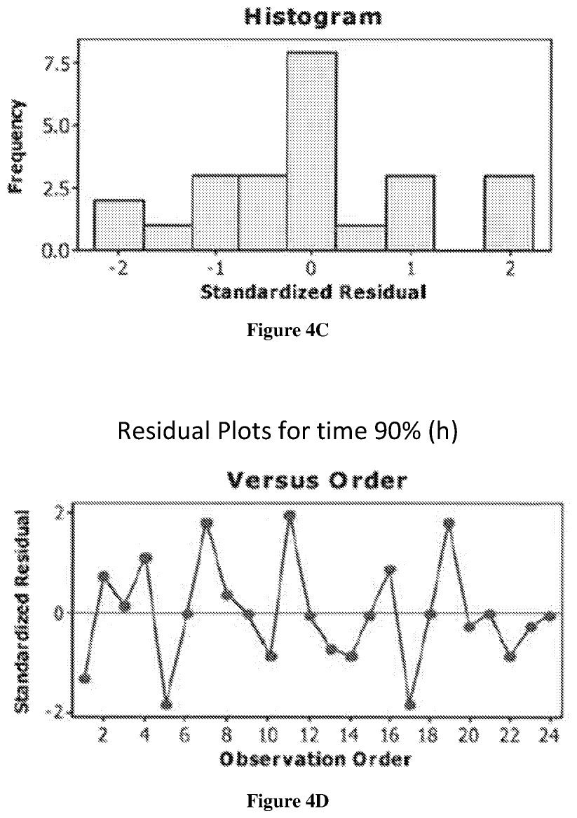

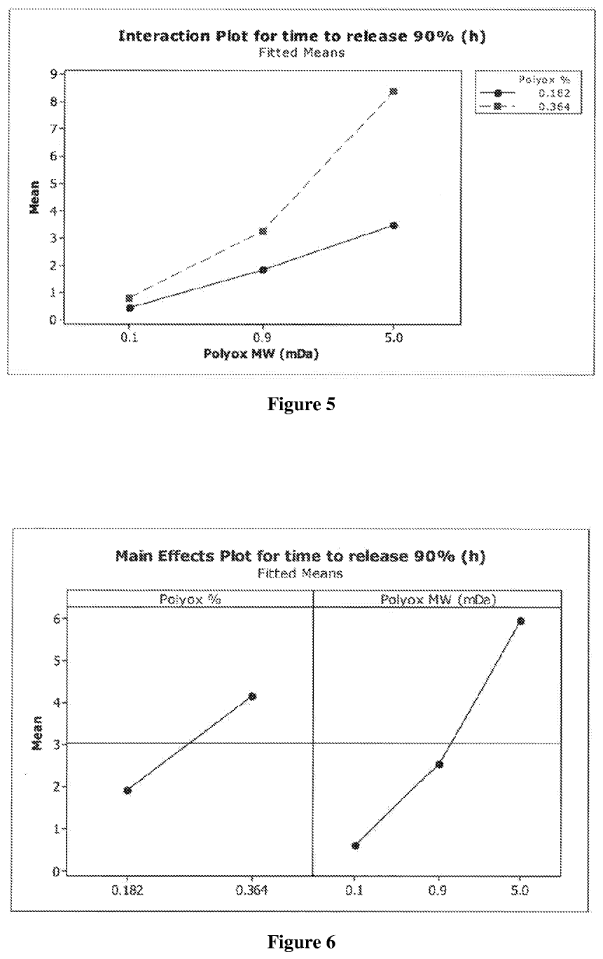

FIG. 4 shows the residual plots for time 90% (hours). FIG. 4A is a normal probability plot, FIG. 4B is a versus fits, FIG. 4C is a histogram, and FIG. 4D is a versus order. FIG. 5 shows the interaction plot for time to release 90% (hours). FIG. 6 is a graph showing the main effect plot for time to release 90% (hours).

Based on these statistical analyses, there is an interaction between the time to release and the PEO molecular weight and concentration. The higher the PEO molecular weight, and the higher the PEO concentration, the slower the API release.

### Example 7—PEO Polymer, High Mn Polyethylene Glycol, and HPMC Polymer Immediate Release Compositions

Immediate release compositions based on PEO resins, high molecular weight polyethylene glycol and low viscosity hydroxypropyl methylcellulose (HPMC) were developed for potential applications in abuse deterrent softgel capsules. The three (3) formulations shown in Table 7 below were prepared. Formulation 13 contained PEO with a number average molecular weight of 100,000 Da and PEG 3350. Formulation 14 contained PEO and HPMC. Formulation 15 contained PEO, PEG 3350, and HPMC.

TABLE 7
Formulations Containing PEG 3350 and HPMC

| | Formulation | | Formulation | | Formulation | |
|---|---|---|---|---|---|---|
| | 13 (g) | % | 14 (g) | % | 15 (g) | % |
| PEO (Mn = | 6.0 | 30.0 | 6.0 | 30.0 | 4.0 | 20.0 |
| 100,000 Da) | | | | | | |
| PEG 400 | 10 | 50.0 | 10 | 50.0 | 10.0 | 50.0 |
| PEG 3350 | 1.0 | 5.0 | — | — | 2.0 | 10.0 |
| HPMC | — | — | 1.0 | 5 | 1.0 | 5.0 |
| METHOCEL ™ | | | | | | |
| VLV | | | | | | |
| Water | 2.0 | 10.0 | 2.0 | 10.0 | 2.0 | 10.0 |
| Diphenhydramine | 1.0 | 5.0 | 1.0 | 5.0 | 1.0 | 5.0 |
| Total (g) | 20.0 | 100.0 | 20.0 | 100.0 | 20.0 | 100.0 |

Size 0 diphenhydramine (DPH) capsules were prepared by mixing PEG 400 with PEO and PEG 3350 and/or HPMC. The DPH was solubilized in water and the DPH solution was added to the PEG/PEO mixture, or the HPMC/PEO mixture, or the PEO/PEG/HPMC mixture. Each capsule was filled with 0.5 g of the fill mixture (25 mg diphenhydramine per capsule). Finally, the capsules were annealed at 60° C. for one (1) hour in an oven.

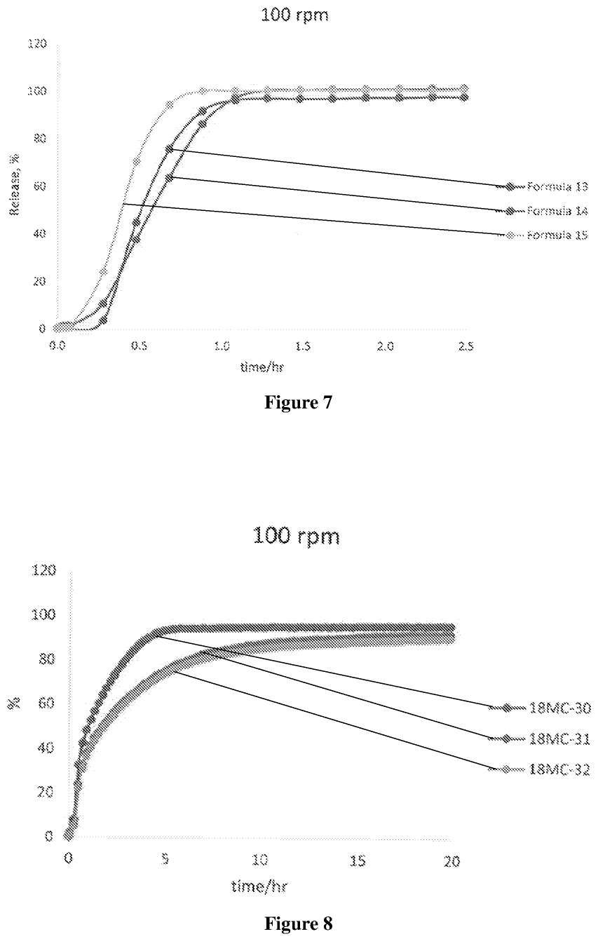

For the dissolution study, fiberoptic dissolution was carried out using USP Apparatus II with paddle speeds of 100 RPM at 37° C. in 500 ml water as the dissolution medium. The dissolution profiles for Formulations 13-15 are shown in FIG. 7.

Formulations 13-15 were shown to be immediate release dosage forms. Diphenhydramine release reached 100% from these formulations in approximately one (1) hour. Formulation 15 had the fastest drug release rate among the three formulations. Not to be bound by theory, but this is believed to have been due to the higher amount of PEG 3350 in Formulation 15.

### Examples 8-10 Controlled Release PEO Softgel Capsules

Three batches of softgel capsules containing fill compositions made from PEO resins with various number average molecular weights (900,000 Da, 5,000,000 Da and 7,000,000 Da) were manufactured using a softgel capsule encapsulation machine. The fill compositions used for batch manufacturing are shown in Tables 8-10 below.

TABLE 8
Fill Formula for Example 8 (18MC-30)

| Mg per capsule | Item Description |
|---|---|
| 25.0 | Diphenhydramine HCl, USP |
| 300.0 | Polyethylene Glycol 400, NF |
| 175.0 | Polyethylene oxide - Mn 900,000 Da |
| | (Polyox ™ WSR 1105) |
| Total | |
| 500.0 | |

TABLE 9
Fill Formula for Example 9 (18MC-31)

| Mg per capsule | Item Description |
|---|---|
| 25.0 | Diphenhydramine HCl, USP |
| 300.0 | Polyethylene Glycol 400, NF |
| 175.0 | Polyethylene oxide - Mn 5,000,000 Da |
| | (Polyox ™ WSR Coagulant) |
| Total | |
| 500.0 | |

TABLE 10
Fill Formula for Example 10 (18MC-32)

| Mg per capsule | Item Description |
|---|---|
| 25.0 | Diphenhydramine HCl, USP |
| 300.0 | Polyethylene Glycol 400, NF |
| 175.0 | Polyethylene oxide - Mn 7,000,000 Da |
| | (Polyox ™ WSR-303) |
| Total | |
| 500.0 | |

After encapsulation, the softgel capsules were sealed in aluminum bags for five (5) days to allow moisture migration from the wet capsule shell into the fill. This moisture migration was utilized to solubilize the PEO in the fill composition, and to form gels to provide sustained release profiles. After five (5) days, the fill moisture of each of the capsules was tested and the results are shown in Table 11 below.

TABLE 11
Softgel Capsule Fill Moisture

| | Fill Moisture, % | | |
|---|---|---|---|
| Example | Sample 1 | Sample 2 | Average |
| 8 (18MC-30) | 19.3 | 16.2 | 17.3 |
| 9 (18MC-31) | 17.1 | 16.2 | 16.7 |
| 10 (18MC-32) | 17.2 | 17.3 | 17.3 |

Although the fill moistures were high enough, the results showed that the PEO resin particles inside the softgel capsules did not fully solubilize. Without being bound by theory, it appears that the PEG 400 bound the fill moisture making it unavailable to fully solubilize the PEO resin particles. Thus, the softgel capsules were annealed at 60° C. for one (1) hour in an oven to melt and solubilize the PEO resin particles. The annealed softgel capsules were then subjected to dissolution tests.

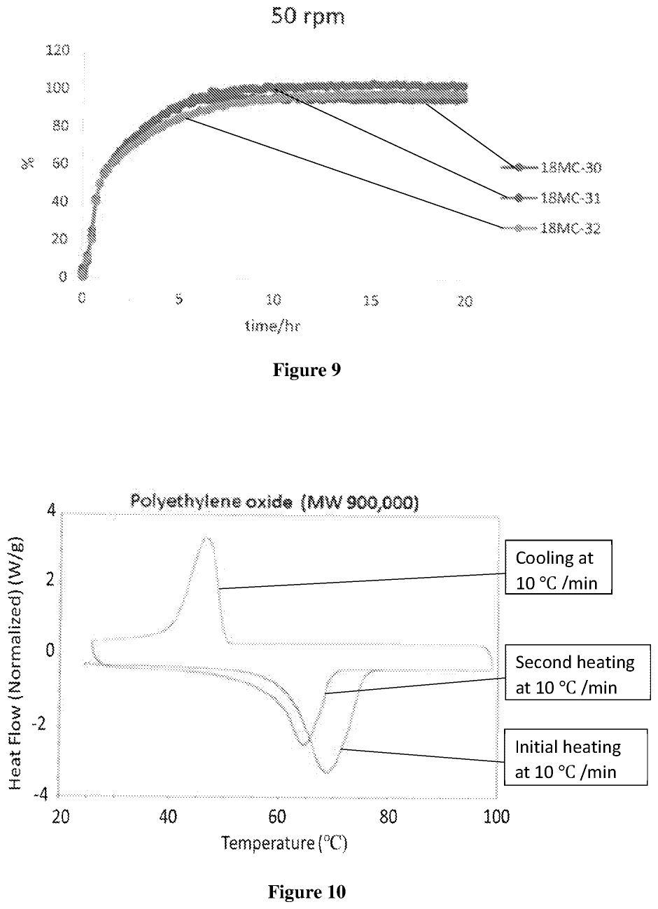

Fiberoptic dissolution using USP Apparatus II with paddle speeds of 50 RPM and 100 RPM at 37° C. in 500 ml water dissolution medium was employed to evaluate the drug release rate in vitro. The comparative dissolution results for capsules prepared with three (3) PEO resins of varying number average molecular weights are shown in FIGS. 8-9.

At the 100 RPM paddle speed, capsules containing PEO with a 900,000 Da number average molecular weight showed a faster drug release rate as compared to capsules prepared with PEO with either a 5,000,000 or a 7,000,000 Da number average molecular weight. The capsules prepared with PEO having the 5,000,000 and 7,000,000 Da number average molecular weights showed similar drug release rates. At 50 RPM, the dissolution profiles were similar for all three of the capsules of Examples 8-10.

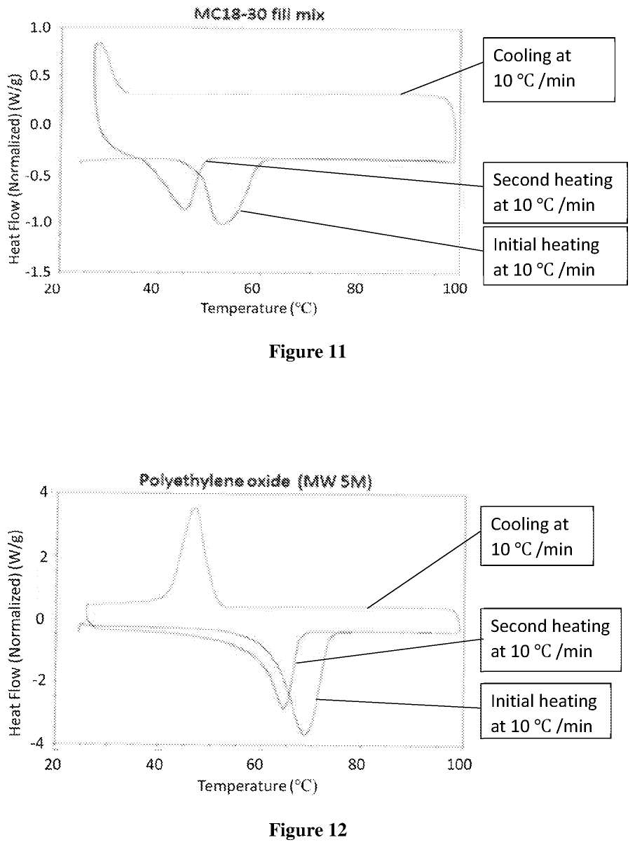

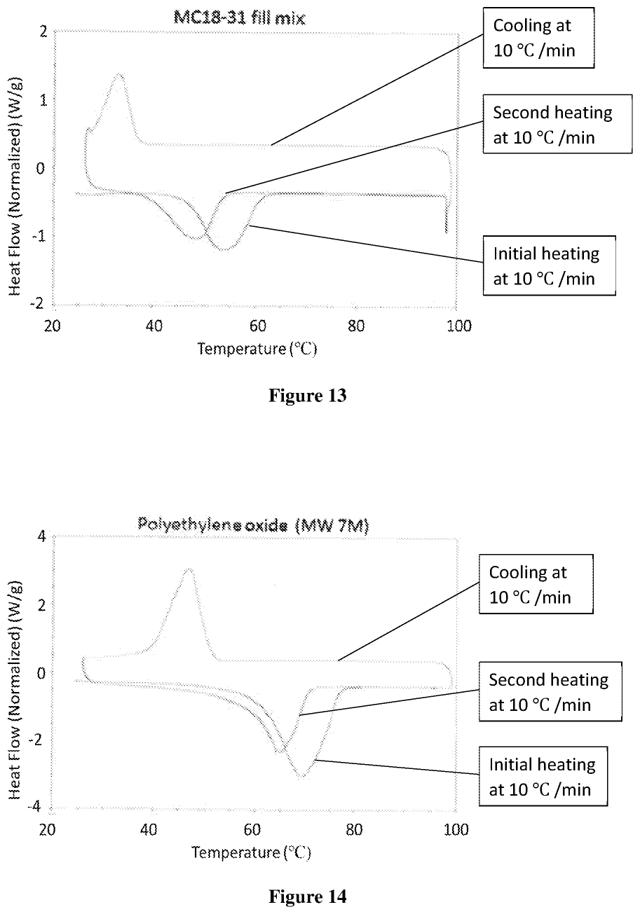

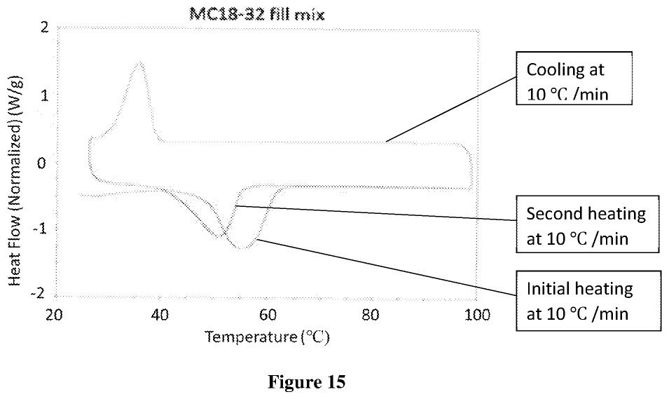

Differential Scanning Colorimetry (DSC) analyses were performed on the PEO resins and the fill compositions used for softgel encapsulation as shown in FIGS. 10-15. The blue curves represent the initial heating at 10° C. per minute. The green curves represent cooling at 10° C. per minute. The red curves represent a second heating at 10° C. per minute. All three PEO resins had melting temperatures below 60° C. upon the initial heating cycle. Not to be bound by theory, this lowered melting temperature of the fill compositions was believed to be due to a plasticizing effect of PEG 400 on the PEO resins. The DSC analyses can be employed to select the proper processing temperature and annealing temperature for the specific fill composition.

Controlled release softgel fill compositions based on polyethylene oxide resins were developed per design of experiment. The effects of PEO concentration and molecular weight on drug release rate were studied. The drug release rate was significantly affected by both the molecular weight of the PEO and the PEO polymer concentration. The higher the PEO molecular weight or the PEO polymer concentration, the slower the drug release rate. The dissolution profiles were similar for the same composition with either a 50 rpm or a 100 rpm paddle speed, indicating that the drug release mechanism was mainly due to diffusion through the polymer matrix.

Compositions containing low molecular weight PEO, PEG 3350 and low viscosity HPMC were also developed for immediate release softgel capsules. These compositions showed immediate release profiles when subjected to dissolution studies.

Three batches of softgel capsules containing various Mn PEO resins were manufactured. The softgel capsules were subjected to dissolution tests. All three batches of softgel capsules show extended release profiles. DSC analyses were performed on the PEO resins and the three compositions. PEG 400 in the composition appears to act as a plasticizing agent to PEO resins, resulting in lower melting temperatures (<60° C.) for the PEO resins, which is beneficial for product manufacture.

### Viscosity Adjustment of Fill Compositions Using Polyethylene Oxide

Three compositions containing only polyethylene oxide (Polyox™) and polyethylene glycol 400 were made to demonstrate how the viscosity of the fill compositions can be controlled by varying the amounts of polyethylene oxide and polyethylene glycol in the fill compositions. The fill compositions and their viscosities are shown in Table 12 below.

TABLE 12
Viscosity Adjustment

| PEO (wt. %) | PEG 400 wt. % | Viscosity (cP) |
|---|---|---|
| 10 | 90 | 229 |
| 30 | 70 | 2374 |
| 40 | 60 | 18190 |

It is to be understood, however, that even though numerous characteristics and advantages of the present disclosure have been set forth in the foregoing description, together with details of the structure and function of the disclosure, the disclosure is illustrative only, and changes may be made in detail, especially in matters of shape, size and arrangement of parts within the principles of the disclosure to the full extent indicated by the broad general meanings of the terms in which the appended claims are expressed.

1. A controlled release capsule fill composition comprising:
(i) an active pharmaceutical ingredient;
(ii) polyethylene oxide having a number average molecule weight of from 0.05 M daltons to 15 M daltons; and
(iii) at least one of water or a hydrophilic carrier having a number average molecule weight of from 50 daltons to 5000 daltons,
wherein either:
(I) the polyethylene oxide is present in an amount of at least 21.5 wt. %, based on a total weight of the controlled release capsule fill composition; or
(II) the hydrophilic carrier is present in an amount of up to 65 wt. %, based on a total weight of the controlled release capsule fill composition.

2. The controlled release capsule fill composition of claim 1, wherein the active pharmaceutical ingredient comprises from about 5 wt. % to about 60 wt. %, based on a total weight of the controlled release capsule fill composition.

3. The controlled release capsule fill composition of claim 1, wherein the polyethylene oxide comprises from 10 wt. % to 65 wt. %, based on a total weight of the controlled release capsule fill composition.

4. The controlled release capsule fill composition of claim 1, wherein the at least one of water or hydrophilic carrier comprises from about 30 wt. % to about 70 wt. %, based on a total weight of the controlled release capsule fill composition.

5. The controlled release capsule fill composition of claim 1, wherein the number average molecule weight of the polyethylene oxide is from about 500,000 daltons to about 15,000,000 daltons.

6. The controlled release capsule fill composition of claim 1, wherein the at least one of water or hydrophilic carrier comprises from 40-60 wt. %, based on a total weight of the controlled release capsule fill composition.

7. The controlled release capsule fill composition of claim 1, wherein the hydrophilic carrier is selected from the group consisting of polyethylene glycol, polypropylene glycol, acetic acid, formic acid, other hydrophilic solvents and combinations thereof

8. The controlled release capsule fill composition of claim 1, wherein the polyethylene oxide comprises from 25-40 wt. %, based on a total weight of the controlled release capsule fill composition.

9. A capsule comprising:
(a) a softgel capsule shell or a hard-capsule shell: and
(b) the controlled release fill composition of claim 1 encapsulated in the softgel capsule shell or hard capsule shell.

10. The capsule of claim 9, wherein less than 80% of the active pharmaceutical ingredient is released after 0.5 hours in a fiberoptic dissolution test using USP Apparatus II at a paddle speed of 100 rpm at 37° C. in 500 ml of 0.1N HCl or water.

11. A method for producing a softgel capsule, said method comprising steps of:
(a) mixing a liquid fill composition comprising:
(i) an active pharmaceutical ingredient;
(ii) polyethylene oxide having a number average molecule weight of from about 0.05 M daltons to about 15 M daltons;
(iii) optionally, one or more additional release rate controlling polymers, and
(iv) at least one of water or a hydrophilic carrier having a number average molecule weight from 200 daltons to 5000 daltons,
wherein either:
(I) the polyethylene oxide is present in an amount of at least 21.5 wt. %, based on a total weight of the fill composition; or
(II) The hydrophilic carrier is present in an amount of up to 65 wt. %, based on a total weight of the fill composition;
(b) encapsulating the mixed liquid fill composition from step (a) in a softgel capsule shell to provide the softgel capsule; and
(c) annealing the softgel capsule to a temperature of from about 40° C. to about 80° C. for a period from about 10 minutes to about 180 minutes to form a solid or semi-solid solution fill composition inside said softgel capsule shell.

12. The method of claim 11, wherein the active pharmaceutical ingredient comprises from about 5 wt. % to about 60 wt. %, based on a total weight of the fill composition and the active pharmaceutical ingredient is classified in one of Biopharmaceutics Classification System Classes I, II, III and IV.

13. The method of claim 11, wherein the fill composition comprises the one or more release rate controlling polymers.

14. The method of claim 11, wherein the polyethylene oxide comprises from 10 wt. % to 65 wt. %, based on a total weight of the fill composition.

15. The method of claim 11, wherein the hydrophilic carrier comprises from about 30 wt. % to about 70 wt. %, based on a total weight of the fill composition.

16. The method of claim 11, wherein the number average molecule weight of the polyethylene oxide is from 1,000,000 to 8,000,000 daltons.

17. The method of claim 11, wherein the hydrophilic carrier comprises from 40-60 wt. %, based on a total weight of the fill composition.

18. The method of claim 11, wherein the polyethylene oxide comprises from 25-40 wt. %, based on a total weight of the fill composition.

19. The method of claim 11, further comprising a step of drying the softgel capsule prior to step (c).

20. A softgel capsule made by the method of claim 11, wherein less than 80% of the active pharmaceutical ingredient is released after 0.5 hours in a fiberoptic dissolution test using USP Apparatus II at a paddle speed of 100 rpm at 37° C. in 500 ml of 0.1 N HCl or water.

21. The capsule according to claim 9,
wherein the capsule is substantially free of flowability enhancing agents selected from the group consisting of glyceryl monocaprylate, glyceryl monocaprylcaprate, glyceryl monolinoleate, oleic acid and magnesium stearate.

22. (canceled)

23. (canceled)

24. (canceled)

25. A method for tuning the dissolution profile of a controlled release fill composition, the method comprising:
adjusting at least one of i)-v) to attain a target dissolution profile of the API:
i) a number average molecular weight of a polyethylene oxide in the controlled release fill composition;
ii) a concentration of a polyethylene oxide in the controlled release fill composition;
iii) a water or hydrophilic carrier content in the controlled release fill composition;
iv) an annealing temperature; and
v) an annealing duration.

26. (canceled)

27. The controlled release capsule fill composition according to claim 1:
wherein a weight ratio of (ii) to (iii) ranges from about 10:1 up to 1:3.

28. (canceled)

29. (canceled)

30. (canceled)

31. (canceled)

32. (canceled)

33. The method according to claim 11
wherein the weight ratio of (ii) to (iv) ranges from about 10:1 up to 1:3.

34. (canceled)
